(19) United States
(12) Patent Application Publication
(10) Pub. No.: US 2010/0184991 A1
(43) Pub. Date: Jul. 22, 2010

(54) PREPARATION OF TERAZOLE DERIVATIVES

(76) Inventors: Gottfried Sedelmeier, Schallstadt (DE); Valentina Aureggi, Como (IT)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 12/749,025

(22) Filed: Mar. 29, 2010

Related U.S. Application Data
(63) Continuation of application No. 11/995,862, filed on Jan. 16, 2008, now abandoned.

(30) Foreign Application Priority Data
Jul. 18, 2005 (GB) 0514686.5

(51) Int. Cl. C07F 15/00; C07D 403/04

(52) U.S. Cl. 548/101; 548/253; 548/254; 548/165

(57) ABSTRACT

The invention relates to a process for the preparation of (S)-pyrrolidine-1H-tetrazole derivatives of formula

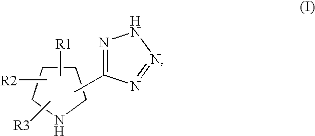

wherein
R₁, R₂ and R₃, independently of one another, represent hydrogen, halogen or an organic radical, in racemic form or as an enantiomer, a tautomer, an analog thereof or a salt thereof.

This application is a continuation of application Ser. No. 11/995,862, filed Jan. 16, 2008, which is a National Phase application of PCT/EP06/007000, filed Jul. 17, 2006, which claims benefit of GB 0514,686.5, filed Jul. 18, 2005.

The invention relates to a process for the preparation of a pyrrolidine-1H-tetrazole derivative, in form of a racemate or an enantiomer, or an analog or a salt thereof, a compound obtained according to this process, new reactants and new tetrazole derivatives.

Thus, the present invention relates to a process for the preparation of (S)-pyrrolidine-1H-tetrazole derivatives of formula

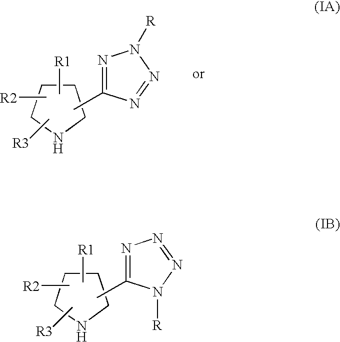

wherein
R₁, R₂ and R₃, independently of one another, represent hydrogen, halogen or an organic radical, and R is hydrogen or is selected from the group consisting of a branched C₃-C₇-alkyl, methyl that can be substituted by one, two or three substituents selected from C₁-C₇-alkyl and C₁-C₇-alkoxy; allyl that can be substituted by one, two or three substituents selected from OH, halo and C₁-C₇-alkoxy, cinnamyl that can be substituted by one, two or three substituents selected from C₁-C₇-alkyl, C₁-C₇-alkoxy and C₂-C₈-alkanoyloxy, C₁-C₃-alkyl that is mono-, di or trisubstituted by phenyl, wherein the phenyl ring is unsubstituted or substituted by one or more, e.g. two or three, substituents e.g. those selected from the group consisting of tert-C₁-C₇-alkyl or C₁-C₇-alkoxy; C₂-C₈-alkanoyloxy; aralkanoyloxy; fluorenyl; silyl such as tri-C₁-C₄-alkyl-silyl, or di-C₁-C₄-alkyl-phenyl-silyl; C₁-C₇-alkyl-sulphonyl; arylsulphonyl such as phenylsulphonyl wherein the phenyl ring is un-substituted or substituted by one or more, e.g. two or three, substituents selected from the group consisting of C₁-C₇-alkyl, C₁-C₇-alkoxy, C₂-C₈-alkanoyl-oxy; C₂-C₈-alkanoyl; benzoyl and esterified carboxy; or R is a cation;
in racemic form or as an enantiomer, a tautomer, an analog thereof or a salt thereof.

Especially, the invention relates to a process for the preparation of (S)-pyrrolidine-1H-tetrazole derivatives of formula

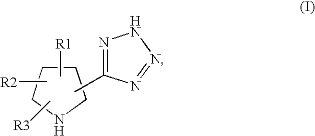

wherein
R₁, R₂ and R₃, independently of one another, represent hydrogen, halogen or an organic radical, in racemic form or as an enantiomer, a tautomer, an analog thereof or a salt thereof.

The general definitions used above and below of the corresponding residues, unless otherwise defined below, have the following meanings:

An organic residue is, for example; an aliphatic residue, an alicyclic residue, a heteroalicyclic residue; an alicyclic-aliphatic residue; a heteroalicyclic-aliphatic residue; a carbocyclic or a heterocyclic aromatic residue; an araliphatic residue or an heteroaraliphatic residue, each residue, independently of one another, being unsubstituted or substituted.

An aliphatic residue is, for example, alkyl, alkenyl or secondarily alkynyl, each of which can be interrupted by NH, substituted NH, O, or S; and each of which can be unsubstituted or substituted, for example, mono-, di- or tri-substituted.

Alkyl is, for example, C₁-C₂₀-alkyl, in particular C₁-C₁₀-alkyl. C₁-C₈-alkyl is preferred, for example methyl, ethyl, n-propyl, isopropyl, n-butyl, isobutyl or tert-butyl.

Alkenyl is, for example, C₃-C₂₀-alkenyl, in particular C₃-C₁₀-alkenyl. Preferred is C₃-C₅-alkenyl; for example, 2-propenyl or 2- or 3-butenyl. Alkenyl is likewise C₂-C₂₀-alkenyl, in particular C₂-C₁₀-alkenyl. Preferred is C₂-C₅-alkenyl.

Alkynyl is, for example, C₃-C₂₀alkynyl, in particular C₃-C₁₀alkynyl. Preferred is C₃-C₅alkynyl such as propargyl. Alkinyl is likewise C₂-C₂₀alkynyl, in particular C₂-C₁₀alkynyl. Preferred is C₂-C₅alkynyl.

Alkyl, alkenyl or alkynyl that can be interrupted by NH, substituted NH, O or S is in particular C₁-C₂₀-alkoxy-C₁-C₂₀-alkyl, —C₃-C₂₀-alkenyl or —C₃-C₂₀-alkynyl, or C₃-C₂₀-alkenyloxy-C₁-C₂₀-alkyl, —C₃-C₂₀-alkenyl or —C₃-C₂₀-alkynyl, for example, C₁-C₁₀-alkoxy-C₁-C₁₀-alkyl, —C₃-C₁₀-alkenyl or —C₃-C₁₀-alkynyl, or C₃-C₁₀-alkenyloxy-C₁-C₁₀-alkyl, —C₃-C₁₀-alkenyl or —C₃-C₁₀-alkynyl. Preferred is C₁-C₇-alkoxy-C₁-C₇-alkyl, —C₃-C₇-alkenyl or —C₃-C₇-alkynyl, or C₃-C₇-alkenyloxy-C₁-C₇-alkyl, —C₃-C₇-alkenyl or —C₃-C₇-alkynyl.

Substituted NH is, for example, NH which is substituted by C₁-C₈-alkyl such as methyl, ethyl or propyl, phenyl-C₁-C₈-alkyl such as benzyl or 2-phenethyl, or by C₁-C₈-alkoxycarbonyl such as carbobenzyloxy or benzyloxycarbonyl or by acyl, such as C₂-C₈-alkyl-alkanoyl, phenyl-C₂-C₅-alkanoyl, benzoyl, C₁-C₈-alkanesulfonyl or benzenesulfonyl.

An alicyclic residue is, for example, mono-, bi- or polycyclic. Preferred is cycloalkyl and secondarily cycloalkenyl, each of which can also be substituted.

Cycloalkyl in particular C₃-C₈cycloalkyl. Preferred is cyclopentyl and cyclohexyl.

Cycloalkenyl is in particular C₃-C₇cycloalkenyl and is preferably cyclopent-2- and -3-enyl, or cyclohex-2- and -3-en-yl.

A heteroalicyclic residue is, for example, an alicyclic residue, wherein at least one carbon atom is replaced by a heteroatom, e.g. NH, substituted NH, O, or S, each of which can also be substituted.

An alicyclic aliphatic residue is, for example, alkyl, alkenyl or alkynyl that is substituted by cycloalkyl or by cycloalkenyl. Preferred is C₁-C₈-alkyl, C₂-C₈-alkenyl or C₂-C₈-alkynyl each of which is substituted by C₃-C₈-cycloalkyl or by C₃-C₈-cycloalkenyl, especially cyclopropylmethyl, cyclopentylmethyl, cyclohexylmethyl, or cyclohexenyl-methyl.

A heterocyclic aliphatic residue is, for example, C₁-C₈-alkyl, C₂-C₈-alkenyl or C₂-C₈-alkynyl each of which substituted by C₃-C₈cycloalkyl or by C₃-C₈-cycloalkenyl wherein one carbon atom of C₃-C₈cycloalkyl or by C₃-C₈-cycloalkenyl, respectively, is replaced by NH, substituted NH, O, or S, especially piperidino-methyl or -ethyl.

A carbocyclic aromatic residue is, for example, a mono- or polycyclic (such as bicyclic) or benzoanellated carbocyclic residue, such as phenyl, naphthyl, but also biphenyl, each of which can also be substituted.

A heterocyclic aromatic residue is, for example, 5- or 6-membered and monocyclic radical which has up to four identical or different hetero atoms, such as nitrogen, oxygen or sulfur atoms, preferably one, two, three or four nitrogen atoms, an oxygen atom or a sulfur atom, each of which can also be substituted. Appropriate 5-membered heteroaryl radicals are, for example, monoaza-, diaza-, triaza-, tetraaza-, monooxa- or monothia-cyclic aryl radicals, such as pyrrolyl, pyrazolyl, imidazolyl, triazolyl, tetrazolyl, furyl and thienyl, while suitable appropriate 6-membered radicals are in particular pyridyl.

An araliphatic residue (aralkyl) is, for example, C₁-C₈-alkyl, C₂-C₈-alkenyl or C₂-C₈-alkynyl each of which is substituted by phenyl or by naphthyl, especially benzyl, 2-phenethyl or 2-phenyl-ethenyl.

A heteroaraliphatic residue, is for example, C₁-C₈-alkyl, C₂-C₈-alkenyl or C₂-C₈-alkynyl each of which is substituted by pyrazolyl, imidazolyl, triazolyl, tetrazolyl, furyl, thienyl or pyridyl, especially pyridylmethyl.

Alkyl, alkenyl, or alkinyl can also be substituted, for example, by a substituent selected from the group consisting e.g. of an alicyclic residue, a heteroalicyclic residue; a carbocyclic and a heterocyclic aromatic residue; each residue, independently of another, being unsubstituted or substituted by one or more, e.g. two or three, substituents, for example, selected from the group consisting of halogen, amino, substituted amino, mercapto, substituted mercapto, hydroxyl, etherified hydroxyl, carboxy, and amidated carboxy.

Alicyclic or heteroalicyclic residues can also be substituted, for example, by one or more, e.g. two or three, substituents selected from the group consisting e.g. of an aliphatic residue, alicyclic residue, a heteroalicyclic residue; a carbocyclic and a heterocyclic aromatic residue; each residue, independently of another, being unsubstituted or substituted by one or more, e.g. two or three, substituents, for example, selected from the group consisting of halogen; amino, substituted amino, mercapto, substituted mercapto, substituted sulfonyl, substituted sulfonyloxy, hydroxyl, etherified hydroxyl, carboxy, and amidated carboxy.

An alicyclic-aliphatic residue, a heteroalicyclic-aliphatic residue, an araliphatic residue or a heteroaraliphatic residue, each residue (e.g. in both the alicyclic and the aliphatic moiety), independently of another, being unsubstituted or substituted by one or more, e.g. two or three, substituents in both structural elements, for example, selected from the group consisting of an aliphatic residue, an alicyclic residue, a heteroalicyclic residue; an alicyclic-aliphatic residue; a heteroalicyclic-aliphatic residue; a carbocyclic aromatic residue, a heterocyclic aromatic residue; an araliphatic residue; an heteroaraliphatic residue, halogen; amino, substituted amino, mercapto, substituted mercapto, substituted sulfonyl, substituted sulfonyloxy, hydroxyl, etherified hydroxyl, carboxy, and amidated carboxy.

A carbocyclic or a heterocyclic aromatic residue can also be substituted, for example, by one or more, e.g. two or three, substituents selected from the group consisting e.g. of an aliphatic residue, alicyclic residue, a heteroalicyclic residue; a carbocyclic and a heterocyclic aromatic residue; each residue, independently of another, being unsubstituted or substituted by one or more, e.g. two or three, substituents, for example, selected from the group consisting of halogen; amino, substituted amino, mercapto, substituted mercapto, substituted sulfonyl, substituted sulfonyloxy, hydroxyl, etherified hydroxyl, carboxy, and amidated carboxy.

Substituents of an aliphatic residue, an alicyclic residue, a heteroalicyclic residue; an alicyclic-aliphatic residue; a heteroalicyclic-aliphatic residue; a carbocyclic or a heterocyclic aromatic residue; an araliphatic residue or an heteroaraliphatic residue, can likewise be acetalized formyl.

Halogen is in particular halogen of atomic number not more than 53, such as fluorine, chlorine, bromine and iodine.

Substituted mercapto is, for example, substituted by an aliphatic residue, an alicyclic residue, a heteroalicyclic residue; an alicyclic-aliphatic residue; a heteroalicyclic-aliphatic residue; a carbocyclic or a heterocyclic aromatic residue; an araliphatic residue or an heteroaraliphatic residue, each residue, independently of another, being unsubstituted or substituted by one or more, e.g. two or three, substituents, for example, selected from the group consisting of halogen; amino, substituted amino, mercapto, substituted mercapto, hydroxyl, etherified hydroxyl, carboxy, and amidated carboxy.

Substituted sulfonyl is, for example, substituted by an aliphatic residue, an alicyclic residue, a heteroalicyclic residue; an alicyclic-aliphatic residue; a heteroalicyclic-aliphatic residue; a carbocyclic or a heterocyclic aromatic residue; an araliphatic residue or an heteroaraliphatic residue, each residue, independently of another, being unsubstituted or substituted by one or more, e.g. two or three, substituents, for example, selected from the group consisting of halogen; amino, substituted amino, mercapto, substituted mercapto, hydroxyl, etherified hydroxyl, carboxy, and amidated carboxy. Preferred is C₁-C₄-alkane-sulfonyl, such as methane-sulfonyl, phenyl-sulfonyl the phenyl ring being unsubstituted or substituted by one or more, e.g. two or three, substituents, selected from the group consisting of e.g. C₁-C₄-alkly, C₁-C₄-alkoxy, halogen and nitro, such as phenylsulfonyl or tosyl.

Substituted sulfonyloxy is, for example, substituted by an aliphatic residue, an alicyclic residue, a heteroalicyclic residue; an alicyclic-aliphatic residue; a heteroalicyclic-aliphatic residue; a carbocyclic or a heterocyclic aromatic residue; an araliphatic residue or an heteroaraliphatic residue, each residue, independently of another, being unsubstituted or substituted by one or more, e.g. two or three, substituents, for example, selected from the group consisting of halogen; amino, substituted amino, mercapto, substituted mercapto, hydroxyl, etherified hydroxyl, carboxy, and amidated carboxy. Preferred is C₁-C₄-alkane-sulfonyl, such as methane-sulfonyloxy, phenyl-sulfonyloxy the phenyl ring being unsubstituted or substituted by one or more, e.g. two or three, substituents, selected from the group consisting of e.g. C₁-C₄-alkly, C₁-C₄-alkoxy, halogen and nitro, such as phenylsulfonlyoxy or tosyloxy.

Etherified hydroxy is, for example, hydroxy etherified by an aliphatic, an alicyclic, heteroalicyclic, an araliphatic, a heteroaryl-aliphatic, a carbocyclic aromatic or heteroaromatic alcohol, each of which can also be substituted.

Esterified carboxy is, for example, carboxy which is esterified by an alcohol which is derived from an aliphatic or araliphatic hydrocarbon radical, such as alkyl, phenyl-alkyl, alkenyl and secondarily alkynyl, and which may be interrupted by —O—, such as alkoxy-alkyl, -alkenyl and -alkynyl. Examples which may be mentioned are C₁-C₇alkoxy-, phenyl-C₁-C₇alkoxy-, C₂-C₇alkenyloxy- and C₁-C₇alkoxy-C₁-C₇alkoxy-carbonyl.

Amidated carboxyl is, for example, carbamoyl in which the amino group is unsubstituted or monosubstituted or, independently of one another, disubstituted by an aliphatic or araliphatic hydrocarbon radical or disubstituted by a divalent aliphatic hydrocarbon radical which may be interrupted by O or may be condensed at two adjacent carbon atoms with a benzene ring, in particular alkylene or lower alkyleneoxy-alkylene. Examples of appropriately substituted amino groups which may be mentioned are C₁-C₇alkyl-, C₂-C₇alkenyl-, C₂-C₇alkynyl-, phenyl-C₁-C₇alkyl-, phenyl-C₂-C₇alkenyl-, phenyl-C₂-C₇alkynyl-, di-C₁-C₇alkyl-, N—C₁-C₇alkyl-N-phenyl-C₁-C₇alkyl- and diphenyl-C₁-C₇alkylamino and also quinol-1-yl, isoquinol-2-yl, C₁-C₇alkylene- and C₁-C₇alkyleneoxy-C₁-C₇alkylene-amino.

Alkylene is, for example, C₁-C₁₀alkylene, in particular, C₁-C₇alkylene, for example methylene, ethylene, or 1,5-pentylene. Corresponding alkylene may also be branched.

Substituted amino has the meanings indicated in connection with substituted carbamoyl and is furthermore acylamino, such as C₂-C₈-alkanoyl-, phenyl-C₂-C₅-alkanoyl-, benzoyl-, C₁-C₈-alkanesulfonyl- or benzenesulfonylamino.

Acetalised formyl is, for example, di-alkoxymethyl or oxy-alkyleneoxymethylene. Most preferred is branched oxy-alkylene-oxy-methylene wherein the alkylene group is branched such as oxy-2,3-butylene-oxy-methylene or oxy-2,3-di-methyl-2,3-butylene-oxy-methylene.

Alkanoyl is, for example, C₂-C₁₀alkanoyl and is in particular C₂-C₇alkanoyl, such as acetyl, propionyl, butyryl, isobutyryl or pivaloyl. C₂-C₅alkanoyl is preferred.

Haloalkylsulfamoyl is in particular halo-C₁-C₁₀alkanesulfamoyl and is in particular C₂-C₇ alkanesulfamoyl, for example, trifluoromethane-, difluoromethane-, 1,1,2-trifluoroethane- or heptafluoropropanesulfamoyl. Halo-C₁-C₄alkanesulfamoyl is preferred.

Pyrrolyl is, for example, 2- or 3-pyrrolyl. Pyrazolyl is 3- or 4-pyrazolyl. Imidazolyl is 2- or 4-imidazolyl. Triazolyl is, for example, 1,3,5-1H-triazol-2-yl or 1,3,4-triazol-2-yl. Tetrazolyl is, for example, 1,2,3,4-tetrazol-5-yl, furyl is 2- or 3-furyl and thienyl is 2- or 3-thienyl, while suitable pyridyl is 2-, 3- or 4-pyridyl or corresponding N-oxido-pyridyl.

Alkoxy is, for example, C₁-C₂₀alkoxy, in particular C₁-C₁₀alkoxy. Preferred is C₁-C₇alkoxy, most preferred C₁-C₄alkoxy such as methoxy, ethoxy, n-propyloxy or tert-butyloxy.

Substituents of residues as mentioned above and below should preferably not comprise those substituents that interfere with the reactants.

Preferred R₁, R₂ or R₃, respectively, is selected from the group consisting of unsubstituted amino, mono and disubstituted amino, azido, alkyl, substituted aryl, substituted heteroaryl, subst. and unsubst. benzyl, alkoxycarbonyl, alkoxy, silyloxy, cyano, sulfonyl, substituted mercapto, especially representatives as specified hereinbefore under corresponding definitions.

Preferably, R is in one embodiment hydrogen

Alternatively, R is an organic residue as defined herein, and is preferably selected from the group consisting of
a branched C₃-C₇-alkyl such as isopropyl or tert-butyl,
methyl that can be substituted by one, two or three substituents selected from C₁-C₇-alkyl and C₁-C₇-alkoxy for example 1-ethoxyethyl, 1-methoxy-1-methylethyl;
allyl that can be substituted by one, two or three substituents selected from OH, halo and C₁-C₇-alkoxy, preferably allyl;
cinnamyl that can be substituted by one, two or three substituents selected from C₁-C₇-alkyl, C₁-C₇-alkoxy and C₂-C₈-alkanoyloxy, preferably cinnamyl;
C₁-C₃-alkyl that is mono-, di or trisubstituted by phenyl, such as benzyl, benzhydryl, trityl, or 1-methyl-1-phenylethyl (cumyl), wherein the phenyl ring is unsubstituted or substituted by one or more, e.g. two or three, substituents e.g. those selected from the group consisting of tert-C₁-C₇-alkyl or C₁-C₇-alkoxy;
C₂-C₈-alkanoyloxy;
aralkanoyloxy;
fluorenyl;
silyl such as tri-C₁-C₄-alkyl-silyl, for example, trimethylsilyl, triethylsilyl or tert-butyl-dimethylsilyl, or di-C₁-C₄-alkyl-phenyl-silyl, for example, dimethyl-phenylsilyl; C₁-C₇-alkyl-sulphonyl;
arylsulphonyl such as phenylsulphonyl wherein the phenyl ring is un-substituted or substituted by one or more, e.g. two or three, substituents selected from the group consisting of C₁-C₇-alkyl, C₁-C₇-alkoxy, C₂-C₈-alkanoyl-oxy; C₂-C₈-alkanoyl such as acetyl or valeroyl; benzoyl and esterified carboxy.

Alternatively, R is a cation. Examples include an alkali metal or an earth alkali metal, for example Li(I), Na(I), K(I), Rb(I), Cs(I), Mg(II), Ca(II), AI(III), Pd (II), Pt(II), Cu(I), Cu(II) and Sr(II), most preferably, Na (I), K(I), Cs(I) or Pd(II). When R is a cation, then the compound of the present invention can also be represented as follows

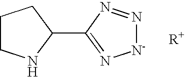

wherein
R₁, R₂ and R₃ are as defined herein.

Most preferred examples of R include H, tert-butyl, methyl, isopropyl, benzyl, benzhydryl, trityl, p-methoxybenzyl, 3,4-dimethoxybenzyl, 1-methyl-1-phenylethyl, triphenylmethyl, (p-methoxyphenyl)-diphenylmethyl, benzyloxymethyl, allyl and cinnamyl, as well as substituted derivatives thereof as described above, in particular phenyl substituted derivatives.

As the compounds of formula (I) have at least one acid group, i.e. the 5-tetrazolyl group, a salt with a base can be formed. A suitable salt with bases is, for example, metal salts, or a salt with ammonia or an organic amine. A corresponding internal salt may furthermore be formed. Compounds of type (I) exist in a zwitterionic form as was confirmed by X-ray crystal structure analysis. As the compounds of formulae (I) have, for example, at least one basic centre, can be an acid addition salt. This is formed, for example, with a strong inorganic acid, with a strong organic carboxylic acid, or with an organic sulfonic acid.

Compound of formula (I) can be used as organo catalysts in the synthesis of compounds in an enantiomerically or a diastereomerically pure form.

An enantiomerically pure form of a compound of formula (I) is >99% ee.

A diastereomerically pure form of a formula of formula (I) is >99% ee.

A tautomer of a compound of formula (I) is a compound of formula

Preferred are pyrrolidine-1H-tetrazoles of formulae

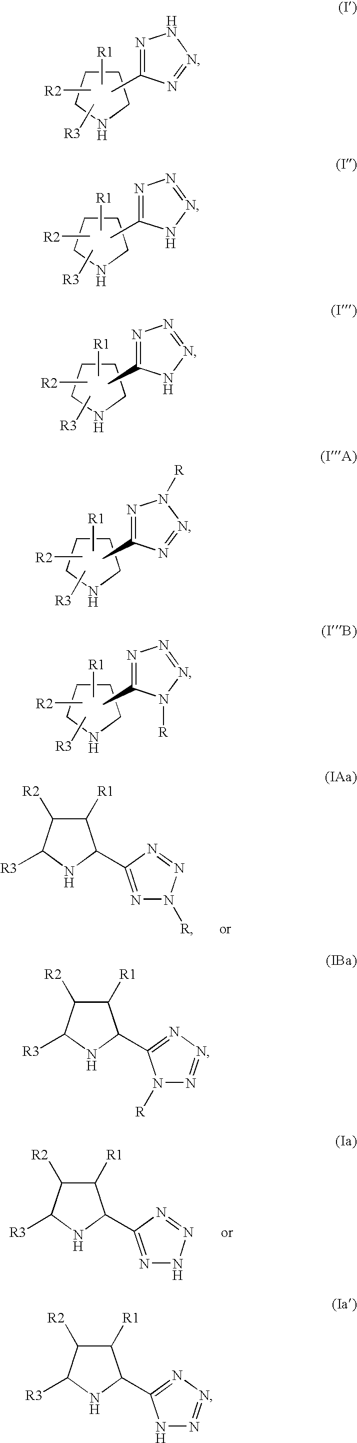

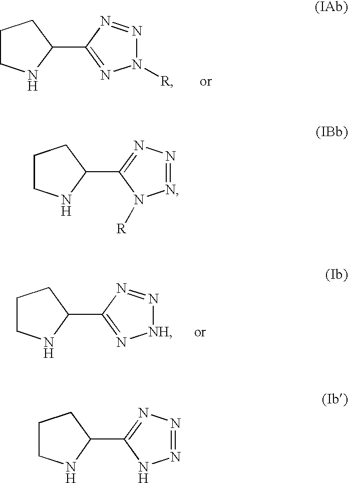

in racemic form or as an enantiomer, as a tautomer, an analog thereof or a salt thereof.

Especially preferred are pyrrolidine-1H-tetrazoles of formulae

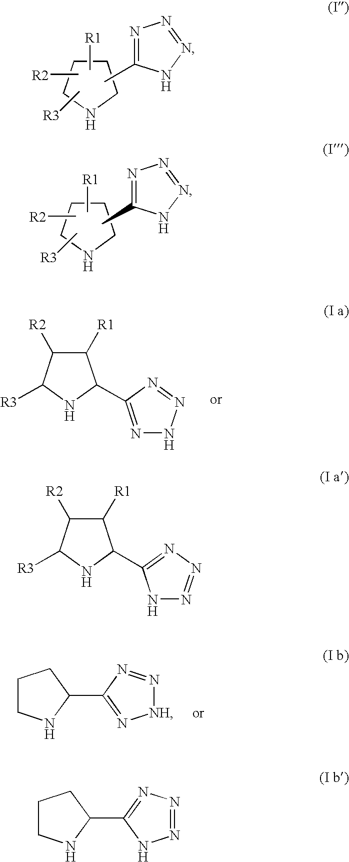

in racemic form or as an enantiomer, as a tautomer, an analog thereof or a salt thereof, which can be used as organo catalysts in the preparation of chiral compounds for example in in aldol reactions, nitroaldol reactions, mannich type reactions, α-amination reactions, α-hydroxylation reactions, michael reactions, and in the synthesis of enantiopure compounds, and will have much broader applications in the synthesis of enantiopure compounds in the future.

Especially preferred is

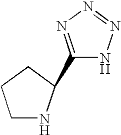

or a tautomer or salt thereof.

Also preferred is a compound of formula

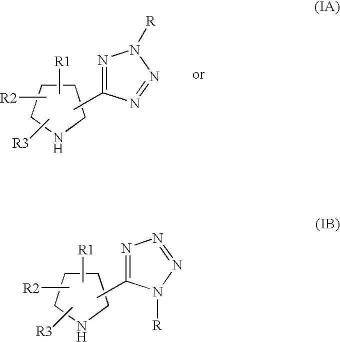

wherein
R₁, R₂ and R₃, independently of one another, represent hydrogen, halogen or an organic radical, and R is hydrogen or tert-butyl, methyl, isopropyl, benzyl, p-methoxybenzyl, 3,4-dimethoxybenzyl, 1-methyl-1-phenylethyl, triphenylmethyl, (p-methoxyphenyl)-diphenylmethyl, benzyloxymethyl, allyl and cinnamyl in racemic form or as an enantiomer, a tautomer, or a salt thereof.

Also preferred is a compound of formula

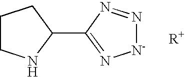

wherein
R₁, R₂ and R₃, independently of one another, represent hydrogen, halogen or an organic radical, and R is a cation, e.g. of an alkali metal or an earth alkali metal, for example Li(I), Na(I), K(I), Rb(I), Cs(I), Mg(II), Ca(II), AI(III), Pd (II), Pt(II), Cu(I), Cu(II) and Sr(II). in racemic form or as an enantiomer or a tautomer thereof.

It is known in the art that tetrazole derivatives can be prepared by reacting various nitriles with organic azides in relatively good yields. Representatives of corresponding azides are, for example, organo-tin azides which have some toxic profile. They have to be handled with special care in production processes, cause ecological problems and require a significant amount of additional process work to recycle them from the wastewater thereby additionally increasing the production costs. Tetrazole forming methods which use trialkylammonium azides or tetraalkylammonium azides may form volatile sublimates in the reaction reactors at higher temperatures which have the risk of explosion and are therefore not easy to handle in large scale production.

There is a strong need to develop process variants, new reagents and intermediates that avoid the above-mentioned disadvantages. Especially, a lot of effort has been made to substitute corresponding organo-tin azides with alternative agents which are viable alternatives in the production of tetrazoles with sufficiently high yields.

The present invention relates to a process for the preparation of (S)-pyrrolidine-1H-tetrazole derivatives of formula

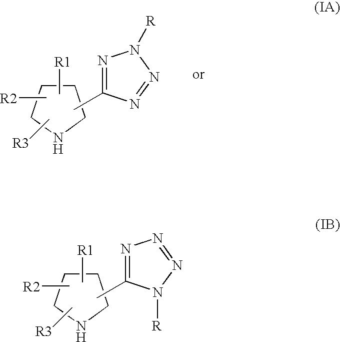

wherein
R₁, R₂ and R₃, independently of one another, represent hydrogen, halogen or an organic radical, and R is hydrogen or is selected from the group consisting of a branched C₃-C₇-alkyl methyl that can be substituted by one, two or three substituents selected from C₁-C₇-alkyl and C₁-C₇-alkoxy; allyl that can be substituted by one, two or three substituents selected from OH, halo and C₁-C₇-alkoxy, cinnamyl that can be substituted by one, two or three substituents selected from C₁-C₇-alkyl, C₁-C₇-alkoxy and C₂-C₈-alkanoyloxy, C₁-C₃-alkyl that is mono-, di or trisubstituted by phenyl, wherein the phenyl ring is unsubstituted or substituted by one or more, e.g. two or three, substituents e.g. those selected from the group consisting of tert-C₁-C₇-alkyl or C₁-C₇-alkoxy, C₂-C₈-alkanoyloxy; aralkanoyloxy; fluorenyl; silyl such as tri-C₁-C₄-alkyl-silyl, or di-C₁-C₄-alkyl-phenyl-silyl; C₁-C₇-alkyl-sulphonyl; arylsulphonyl such as phenylsulphonyl wherein the phenyl ring is un-substituted or substituted by one or more, e.g. two or three, substituents selected from the group consisting of C₁-C₇-alkyl, C₁-C₇-alkoxy, C₂-C₈-alkanoyl-oxy; C₂-C₈-alkanoyl; benzoyl and esterified carboxy; or R is a cation;
in racemic form or as an enantiomer, a tautomer, an analog thereof or a salt thereof,
comprising
(i) reacting a compound of formula

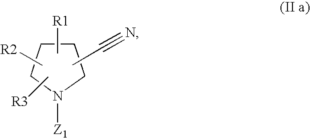

wherein variables R₁, R₂ and R₃ have the meaning as defined above, with an azide of formula (R₄)(R₅)M-N₃ (II b), wherein R₄ and R₅, independently of another, represent an organic residue such as an aliphatic residue, an alicyclic residue, a heteroalicyclic residue; an alicyclic-aliphatic residue; a heteroalicyclic-aliphatic residue; a carbocyclic or a heterocyclic aromatic residue; an araliphatic residue or an heteroaraliphatic residue, each residue, independently of another, being unsubstituted or substituted; and M is boron or aluminium; and Z₁ represents a protecting group, and
(ii) isolating the resulting compound of formula (IA) or (IB).

The present invention also relates to a process for the manufacture of pyrrolidine-1H-tetrazole derivative of formula

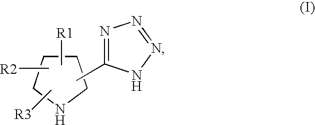

in racemic form or as an enantiomer, as a tautomer, an analog thereof or a salt thereof,
wherein R represents an organic residue;
comprising
(i) reacting a compound of formula

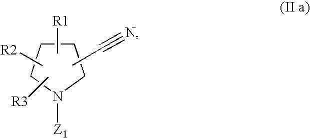

wherein variables R₁, R₂ and R₃ have the meaning as defined above, with an azide of formula (R₄)(R₅)M-N₃ (II b), wherein R₄ and R₅, independently of another, represent an organic residue such as an aliphatic residue, an alicyclic residue, a heteroalicyclic residue; an alicyclic-aliphatic residue; a heteroalicyclic-aliphatic residue; a carbocyclic or a heterocyclic aromatic residue; an araliphatic residue or an heteroaraliphatic residue, each residue, independently of another, being unsubstituted or substituted; and M is boron or aluminium; and Z₁ represents a protecting group, and
(ii) isolating the resulting compound of formula (I).

A protecting group Z₁ is, for example, an amino protecting group which is conventionally used in peptide chemistry (cf.: “Protective groups in Organic Synthesis”, 5^{th}. Ed. T. W. Greene & P. G. M. Wuts), especially in chemistry of protecting pyrrolidines. Preferred protecting groups comprise, for example, (i) C₁-C₂-alkyl that is mono-, di- or trisubstituted by phenyl, such as benzyl, (or) benzhydryl or trityl, wherein the phenyl ring is unsubstituted or substituted by one or more, e.g. two or three, residues e.g. those selected from the group consisting of C₁-C₇-alkyl, hydroxy, C₁-C₇-alkoxy, C₂-C₈-alkanoyl-oxy, halogen, nitro, cyano, and CF₃; phenyl-C1-C2-alkoxycarbonyl; and allyl or cinnamyl.

Especially preferred are benzyloxycarbonyl (Cbz), 9-fluorenylmethyloxycarbony (Fmoc), benzyloxymethyl (BOM), pivaloyl-oxy-methyl (POM), trichloroethxoycarbonyl (Troc), 1-adamantyloxycarbonxyl (Adoc), but can also be benzyl, cumyl, benzhydryl, trityl, allyl. Z₁ can also be silyl, like trialklysilyl, especially trimethylsilyl, tert.-butyl-dimethylsilyl, triethylsilyl, triisopropylsilyl, trimethylsilyethoxymethyl (SEM), and can also be substituted sulfonyl or substituted sulfenyl. Substituted sulfonyl toluolsulfonylchlorid (Tos-Cl). Substituted sulfenyl

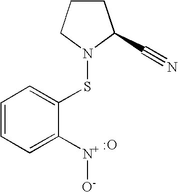

Preferably, a compound of formula (II a) is used, wherein substituents of variables R₁, R₂ or R₃, respectively, do not interfere during the reaction with a compound of formula (II b).

A preferred azide of formula (R₄)(R₅)M-N₃ (II b) is a corresponding compound, wherein M is aluminium or boron, R₄ and R₅, independently of one another, is C₁-C₈-alkyl such as methyl, ethyl, propyl, i-propyl, diisobutyl, tert-butyl or n-octyl; C₃-C₇alkenyl such as allyl or crotyl, C₃-C₇-cycloalkyl such as cyclohexyl; phenyl-C₁-C₄-alkyl such as benzyl or 2-phenethyl; phenyl-C₃-C₅alkenyl such as cinnamyl, or C₃-C₈-cycloalkyl-C₁-C₈-alkyl such as cyclopropylmethyl or cyclohexylmethyl. Likewise, R₄ and R₅, independently of one another, is phenyl-C₂-C₅alkenyl.

Especially preferred azides are those as mentioned in the Examples.

The molar ratio of an azide of formula (II b) and a nitrile of formula (II a) is in a range from 5 to 1, preferably, from 3 to 1, most preferably, from 1.8 to 1 or from 1.2 to 1.

An inert solvent, diluent or mixture thereof should be selected which means that it cannot react with the starting material or intermediates. A suitable solvent is, for example, selected from the group consisting of aliphatic, cycloaliphatic and aromatic hydrocarbon, such as an C₅-C₁₀-alkane e.g. heptane, a cycloalkane such as cyclohexane; and alkylated C₃-C₇cycloalkane such as methyl-cyclohexane or 1,3-dimethyl-cyclohexane, an alkylated benzene such as ethylbenzene, toluene, xylene, cumene, or mesitylene; a halogenated aromatic solvent such as chlorobenzene, o-, m- or p-chlorotoluene, dichlorobenzene, and trifluoromethylbenzene which may be further substituted e.g. by C₁-C₇alkyl or C₁-C₇alkoxy; and a halogenated hydrocarbon, for example, a halogenated aromatic compound, such as chlorobenzene. A further solvent may be an ether, such as tetrahydrofurane. Furthermore, a suitable solvent, diluent or mixture thereof should have a boiling point that is high enough to be used under the reaction conditions.

Preferred solvents or diluents are aliphatic hydrocarbons, for example, C₆-C₉alkanes such as heptane or n-octane; aromatic hydrocarbons, for example, phenyl substituted by C₁-C₄alkyl such as toluene or xylene, or mixtures thereof.

The reaction temperature is preferred in the temperature range of from room temperature to the boiling point of the solvent, diluent or mixture thereof, for example, a reaction temperature range is from about 20° C. to about 170° C., preferably, from about 60° C. to about 130° C. or to about 140° C., depending on the reactivity and combination of the reactants. A person skilled in the art is fully enabled to select corresponding suitable solvent and diluent systems and reaction conditions adapted to the choice of the solvent system and reactants.

The reaction is most preferably carried out under anhydrous conditions.

In a preferred embodiment of the present invention, the invention is carried out in a temperature range of from 80° C. to 120° C., preferably between 60° C. and 80° C. or especially in the case of some labile protecting groups preferably between 30° and 50° C.

The isolation step is carried out according to conventional isolation methods, such as by crystallizing the resulting compound of formula (I) or a tautomer or salt thereof, from the reaction mixture or by chromatography of the reaction mixture, such as by crystallizing the resulting compound from the reaction mixture—if desired or necessary after work-up, especially by extraction—or by chromatography of the reaction mixture. Reference in this context is also made to the working examples.

A variant of the process of the present invention for the manufacture of a compound of formula (I), in racemic form or as an enantiomer, as a tautomer, an analog thereof or a salt thereof, wherein R represents an organic residue;

comprises
(a) reacting a compound of formula

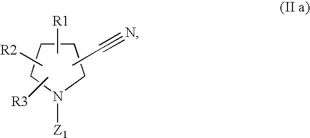

wherein variables R₁, R₂ and R₃ have the meaning as defined above and Z₁ is a protecting group,
with an azide of formula (R₄)(R₅)M-N₃ (II b), wherein R₄ and R₅ have the meaning as defined above,
(b) splitting off the protecting group in a resulting compound of formula (II c)

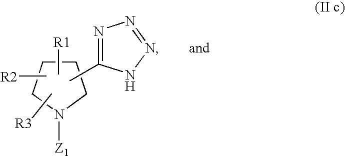

(c) isolating the resulting compound of formula (I).

In a variant of the present invention, when R is selected from the group consisting of a branched C₃-C₇-alkyl methyl that can be substituted by one, two or three substituents selected from C₁-C₇-alkyl and C₁-C₇-alkoxy; allyl that can be substituted by one, two or three substituents selected from OH, halo and C₁-C₇-alkoxy, cinnamyl that can be substituted by one, two or three substituents selected from C₁-C₇-alkyl, C₁-C₇-alkoxy and C₂-C₈-alkanoyloxy, C₁-C₃-alkyl that is mono-, di or trisubstituted by phenyl, wherein the phenyl ring is unsubstituted or substituted by one or more, e.g. two or three, substituents e.g. those selected from the group consisting of tert-C₁-C₇-alkyl or C₁-C₇-alkoxy, C₂-C₈-alkanoyloxy; aralkanoyloxy; fluorenyl; silyl such as tri-C₁-C₄-alkyl-silyl, or di-C₁-C₄-alkyl-phenyl-silyl; C₁-C₇-alkyl-sulphonyl; arylsulphonyl such as phenylsulphonyl wherein the phenyl ring is un-substituted or substituted by one or more, e.g. two or three, substituents selected from the group consisting of C₁-C₇-alkyl, C₁-C₇-alkoxy, C₂-C₈-alkanoyl-oxy; C₂-C₈-alkanoyl; benzoyl and esterified carboxy, then this group can be introduced as follows, preferably after step (i).

For the introduction of suitable groups R into the tetrazole ring in position N-1 or in position N-2 it is possible to use either acidic or basic conditions. These groups can be functional groups because they have a function in the catalytic cycle of organocatalytic reactions or they can be protecting groups and functional groups at the same time. For example, the residue R like t-Bu, cumyl or benzhydryl can be introduced to the tetrazole ring under strong acidic conditions. Suitably, inert solvents are used which are the same as described above for step (i), preferably halogenated hydrocarbons such as dichloromethane. Acids which can be used are trifluoroacetic acid, methanesulfonic acid, conc. hydrochloric acid, sulfuric acid, etc.

During the introduction of this groups a mixture of isomers are observed (N-1 and N-2 position) according to the tautomers in the starting material. However when big groups like t-Bu or cumyl, benzhydryl or trityl groups are introduced the major product is the N-2 isomer. In contrast to this a different ratio mixture is obtained when basic conditions are used.

Basic conditions are particularly preferred to introduce smaller groups like methyl ethyl, isopropyl, allyl or benzyl. This groups are preferably introduced before a protecting group, like Cbz or Boc, at the pyrrolidine ring is split off. Basic conditions are for example an inorganic base such as potassium carbonate in an inert solvent. Suitably, inert solvents are used which are the same as described above for step (i), preferably acetonitrile, THF, dioxane, DMF or NMP. The corresponding alkylating agent like R—X, wherein X is halo, such as I, can be added at room temperature.

Another method for the introduction of alkyl groups is the use of N-alkyl triazenes, such as R—NH—N═N-tolyl under neutral conditions as described herein.

The respective compound of the present invention wherein R is a cation can be formed from the unsubstituted compound of formula (I), i.e. wherein R is hydrogen, and using the corresponding base such as NaOH, NaOMe, KOH, Pd(OAc)₂, or CsOH.

In a preferred variant of the present invention, the formation of the tetrazole ring (a) and splitting off of the protecting group (b) can be effected in one step, if suitable reaction conditions are selected. For example, if Z₁ is a Boc-protecting group and if the reaction is carried out in an acidique (<pH 2) aqueous system, the Boc group is split off so that compound (I) can be obtained in a one-pot reaction sequence.

A hydrogenation catalyst is, for example, nickel, such as Raney nickel, and noble metals or their derivatives, for example oxides, such as palladium, platinium or platinum oxide, which may be applied, if desired, to support materials, for example to carbon or calcium carbonate, for example, platinium on carbon. The hydrogenation with hydrogen or a hydrogen donor may preferably be carried out at pressures between 1 and about 100 atmosphere and at room temperature between about −80° to about 200° C., in particular between room temperature and about 100° C.

Methods of splitting off corresponding amino protecting groups are known by the person skilled in the art and are especially described in “Protective groups in Organic Synthesis”, 5^{th}. Ed., T. W. Greene & P. G. M. Wuts. The corresponding methods are herewith incorporated by reference.

Splitting off the protecting group is carried out by using adequate reaction conditions which are to be adapted to the protecting group used. For example, if Z₁ represents benzyloxycarbonyl, said protecting group is split off under hydrogenation, especially in the presence of a hydrogenation catalyst or with an excess of dialkyl aluminium reagent.

The protecting groups can be removed form the pyrrolidin nitrogen either by mild to strong acidic treatment like HCl, HBr, CF₃COOH (Boc, Adoc, Cbz group) or by hydrogenation or transfer hydrogenation in the presence of a catalyst (e.g. BOM, Cbz, Bn, cumyl, etc.) The Fmoc protecting group can be cleaved under mild conditions with bases like morpho-line or piperidine or by tetrabutyl ammonium fluoride at room temperature in short times. Protecting groups like silyl groups can be removed by acid treatment or by treatment with fluoride ions.

Compounds of formula (II a) are either known or can be prepared using methods known in the art.

Preferred are compounds of formula (II a), wherein R₁, R₂, R₃ are n-alkyl, branched alkyl, cycloalkyl, alkoxy, aryl, heteroaryl, aralkyl, benzyl, substituted benzyl, allyl, carboxyalkyl, subst. amino, azido, arylsulfonyl, mercapto, substituted mercapto. Preferred are C₁-C₇-alkyl such as isopropyl, especially 4 isopropyl, phenyl, especially 5-phenyl, cyano, especially 5-cynao, carbo-C₁-C₄-alkoxy, especially 5-carbo-ethoxy, halogen, especially 4-fluoro, furthermore amino, substituted amino, azido, aryl, hydroxyl, esterified hydroxyl, mercapto, substituted mercapto, tri-alkyl-silyloxy.

Preferably, a compound of formula (II a) is used, wherein substituents of variable R do not interfere during the reaction with a compound of formula (II b).

A compound of formula (II a) is preferably a corresponding compound, wherein R is as defined above.

A preferred azide of formula (R₄)(R₅)M-N₃ (II b) is a corresponding compound, wherein M is aluminium or boron, R₄ and R₅, independently of one another, is C₁-C₈-alkyl such as methyl, ethyl, propyl, diisobutyl, tert-butyl or n-octyl; C₃-C₇alkenyl such as allyl or crotyl, C₃-C₇-cycloalkyl such as cyclohexyl; phenyl-C₁-C₄-alkyl such as benzyl or 2-phenethyl; phenyl-C₃-C₅alkenyl such as cinnamyl, or C₃-C₈-cycloalkyl-C₁-C₈-alkyl such as cyclopropylmethyl or cyclohexylmethyl. Likewise, R₄ and R₅, independently of one another, is phenyl-C₂-C₅alkenyl.

Especially preferred azides are those as mentioned in the Examples.

The molar ratio of an azide of formula (II b) and a nitrile of formula (II a) is in a range from 5 to 1, preferably, from 3 to 1, most preferably, from 1.8 to 1 or from 1.2 to 1.

An inert solvent, diluent or mixture thereof should be selected which means that it cannot react with the starting material or intermediates. A suitable solvent is, for example, selected from the group consisting of aliphatic, cycloaliphatic and aromatic hydrocarbon, such as an C₅-C₁₀-alkane e.g. heptane, a cycloalkane such as cyclohexane; and alkylated C₃-C₇cycloalkane such as methyl-cyclohexane or 1,3-dimethyl-cyclohexane, an alkylated benzene such as ethylbenzene, toluene, xylene, cumene, or mesitylene; a halogenated aromatic solvent such as chlorobenzene, o-, m- or p-chlorotoluene, dichlorobenzene, and trifluoromethylbenzene which may be further substituted e.g. by C₁-C₇alkyl or C₁-C₇alkoxy; and a halogenated hydrocarbon, for example, a halogenated aromatic compound, such as chlorobenzene. A further solvent may be an ether, such as tetrahydrofurane. Furthermore, a suitable solvent, diluent or mixture thereof should have a boiling point that is high enough to be used under the reaction conditions.

Preferred solvents or diluents are aliphatic hydrocarbons, for example, C₆-C₉alkanes such as heptane or n-octane; aromatic hydrocarbons, for example, phenyl substituted by C₁-C₄alkyl such as toluene or xylene, or mixtures thereof.

The reaction temperature is preferred in the temperature range of from room temperature to the boiling point of the solvent, diluent or mixture thereof, for example, a reaction temperature range is from about 20° C. to about 170° C., preferably, from about 60° C. to about 130° C. or to about 140° C., depending on the reactivity and combination of the reactants. A person skilled in the art is fully enabled to select corresponding suitable solvent and diluent systems and reaction conditions adapted to the choice of the solvent system and reactants.

The reaction is most preferably carried out under anhydrous conditions.

In a preferred embodiment of the present invention, the invention is carried out in a temperature range of from 0° C. to 120° C., such as 30° C. to 120° C., preferably between 40° C. and 60° C.

The isolation step is carried out according to conventional isolation methods, such as by crystallizing the resulting compound of formula (I) or a tautomer or salt thereof, from the reaction mixture or by chromatography of the reaction mixture, such as by crystallizing the resulting compound from the reaction mixture—if desired or necessary after work-up, especially by extraction—or by chromatography of the reaction mixture. Reference in this context is also made to the working examples.

In a preferred embodiment of the present invention, the (S)- or (R)-enantiomers, respectively, of compounds of formula (I) can be obtained. Either corresponding enantiomers of compounds of formula (II a) are used or the resulting racemates of compounds of formula (I) can be separated into the corresponding enantiomers.

An other preferred embodiment of the present invention is directed to a process for the manufacture of (S)-5-pyrrolidin-2-yl-1H-tetrazole of formula (I b′)

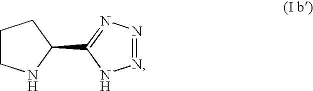

or a tautomer or a salt thereof, comprising
(a) reacting a compound of formula (II a′)

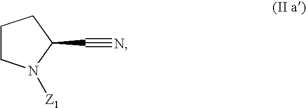

wherein Z₁ is a protecting group, with an azide of formula (R₄)(R₅)M-N₃ (II b), wherein variables R₄, R₅ and M have the meanings as given above and below,
(b) splitting off the protecting group in a resulting in a compound of formula (II c′)

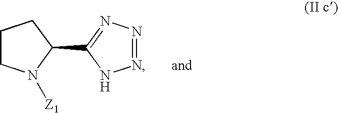

(c) isolating a compound of formula (I b′).

The reaction conditions of steps (a) and (b) could be selected to avoid isolation of a compound of formula (II c).

The present invention is directed to the use of a compound of formulae (I), (I′), (I″), (I′″), (I b), (I b′), (I c) or (I c′) for organo catalysis, e.g. as described in
a) S. Ley et al., Synlett, 2005 (4) 611;
b) H. Yamamoto et al., Proc. Nat. Acad. Sc. 101, 5374 (2004)
c) I. Arvidsson et al., Tetrah. Asymm. 15, 1831 (2004)
d) C. F. Barbas et al., Org. Lett. 7, 867 (2005)
e) A. Cordova et al., Tetrahedron Lett., 46, 3385 (2005).

Compounds of formula (II a) are either known or can be prepared using methods known in the art.

Azides of formula (II b) can be prepared, for example, by reacting a compound of formula (R₁)(R₂)M-X (II c), wherein M is aluminium or boron, R₁ and R₂ have the meanings as defined above and X is a leaving group e.g. halogen, such as fluoride, chloride, bromide or iodide; or a sulphonate, such as an alkane sulfonate e.g. methanesulphonate; a halogenated alkane sulfonate e.g. trifluoromethansulfonate, an aromatic sulphonate e.g. tosylate; with an azide, preferably an alkaline metal azide, such as a lithium, sodium or potassium azide.

The formation of an azide of formula (II b) is carried out, in particular, in the presence of an inert solvent or diluent or a mixture thereof, in a temperature range of 0° C. to 120° C. The reaction is most preferably carried out under anhydrous conditions.

Preferred azides comprise compounds of formula (II b), wherein R₁ and R₂, independently of one another, represent C₁-C₈-alkyl such as ethyl, iso-propyl, n-propyl, n-butyl, sec-butyl, tert-butyl or n-octyl, C₃-C₈-cycloalkyl, C₃-C₈-cycloalkyl-C₁-C₈-alkyl or aryl-C₁-C₈-alkyl such as benzyl or 2 phenethyl; and M is boron or aluminium. Corresponding representatives are dimethyl aluminium azide, diethyl aluminium azide, diisopropyl aluminium azide, dipropyl aluminium azide, diisobutyl aluminium azide, dibutyl aluminium azide, dicyclohexyl aluminium azide, diethyl boron azide, diisopropyl boron azide, dipropyl boron azide, diisobutyl boron azide, dibutyl boron azide or dicyclohexyl boron azide, furthermore diaryl boron azide such as diphenyl boron azide.

It might be that, dependent on the kind of substituents, reactive substituents could also react with the azide. For example, an aromatic hydroxy group or a benzylic hydroxyl group may react with an azide of formula (II b), however, the resulting hydroxy function masked by a metal or by an organo metal group can be split with e.g. an acid resulting in a compound of formula (I); accordingly, in this situation, a higher amount of a compound of formula (II a) needs to be used. An ester group might form an acyl-azide with a compound of formula (II b), while an epoxy ring structure might be opened with an compound of formula (II b). However, the person skilled in the art would be able to either directly anticipate that starting compounds with specific reactive substituents could not be used, as these substituents might react with the azide instead of the cyano function, or the person skilled in the art would, when corresponding side reactions are realized, protect corresponding reactive groups and later on split-off the corresponding protecting groups by using conventional methods known per se.

The invention relates to the compounds obtained according any process of the present invention.

The examples outline specific embodiments of the present invention, but are not to limit the scope of the invention.

### EXAMPLE 1

### (S)-2-(1H-tetrazol-5-yl)-pyrrolidin-1-carboxylic acid benzyl ester

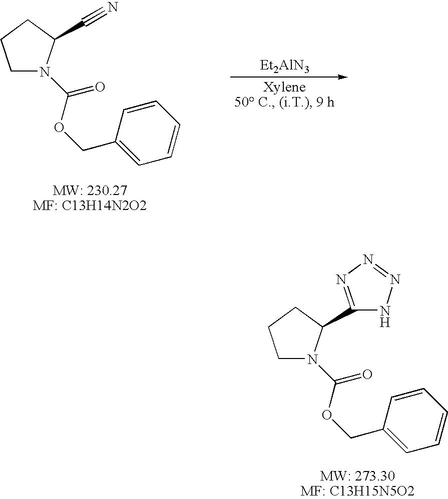

4.875 g of granular sodium azide (75 mmol) are added to a cold solution (0° C.) of 28 ml of diethyl aluminum chloride (75 mmol, 2.7 molar in xylene), and the mixture is stirred at room temperature for 4 to 7 hours. 11.5 g of (S)-2-cyano-pyrrolidine-1-carboxylic acid benzyl ester (50 mmol) are then added as a solid under argon, at 0° C., to the diethylaluminium azide solution, and the mixture is warmed to 54° C. (external temperature), 50° C. (internal temperature), and stirred for 7-9 hours at this temperature. After complete conversion the reaction mixture is added dropwise, at 0° C., to a solution of 85 ml of NaOH (7%; 150 mmol) containing 10.350 g of sodium nitrite (150 mmol). 40 of HCl (6N) are slowly added under cooling at 0° C., to pH 2.5. The product is extracted five times with 50 ml portion of ethyl acetate. The solvent is removed to give the crude residue which is re-dissolved with 80 ml of ethyl acetate, and extracted four times with 60 ml portions of potassium carbonate (10%). The aqueous phase is treated, at 0° C., with HCl (6N) to pH 2.5, and extracted four times with 100 ml portions of ethyl acetate. The solvent is removed to give an oil which solidifies in the refrigerator to a white solid.

Analysis: The reaction is followed via HPLC: a sample of the mixture (c.a. 0.15 ml) is quenched with 0.5 ml of HCl 1N; 0.2 ml of acetonitrile are added, and the organic phase is checked with a HPLC.

M.p.: 84-86° C., onset of exothermic decomposition: 204° C., with maximum at 253° C. EI-MS: 274; 272; 230; HPLC: Hewlett Packard, solvents. H₃PO₄ (1%), acetonitrile; flow: 2 ml/min; injection: 5.0 μl; wavelength 220 nm, 40° C. Column: Merck, Chromolith Performance RP-18e 100-4.6 mm: 5.465 min; TLC: (Eluent: Toluene/Etylacetate/Acetic Acid 20:20:1): R_{f}=0.22; UV: (in acetonitrile, 0.10000 g/L) 236.75 nm min, 257.89 nm max; IR: 3000-2400 cm⁻¹; s1694 cm⁻¹; s1443 cm⁻¹; s1404 cm⁻¹; s1132 cm⁻¹; Raman: s1005 cm⁻¹; ¹HNMR: (500 MHz; DMSO) 2.1 ppm (m, 1H); 2.28 ppm (m, 2H); 2.82 ppm (m, 1H); 3.56 ppm (m, 2H), 4.98 ppm (m, 2H); 5.18 ppm (m, 1H); 7.21 ppm (m, 5H).

### EXAMPLE 2

### 2-(1H-Tetrazol-5-yl)pyrrolidine-1-carboxylic acid tert-butyl ester

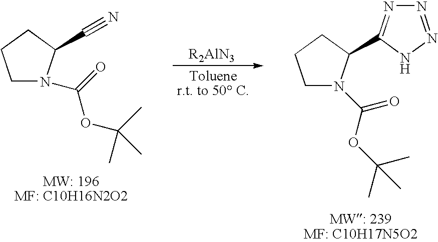

390 mg of granular sodium azide (6 mmol) are added at 0° C. to a solution of 2.4 ml of diethyl aluminium chloride (6 mmol, 2.5 molar in toluene) and the mixture is stirred at room temperature for 4 to 7 hours. 981 mg of 2-cyano-pyrrolidine-1-carboxylic acid t-butyl ester (5 mmol) are added to the diethyl aluminum azide solution at r.t. The mixture is stirred 20 to 30 h at 40° C. After complete conversion the mixture is added dropwise to a cold solution (0° C.) of 25 ml of KHSO₄ (10%) containing 1.24 g of NaNO₂ (18 mmol), and extracted four times with 25 ml portion of ethyl acetate. The solvent is removed to give 1.4 g of the crude product which is dissolved again in 20 ml of ethyl acetate. This phase is washed several times with K₂CO₃ (10%) to extract the product to the aqueous phase as the potassium salt. The basic aqueous phase is carefully treated at 0° C. with KHSO₄ solution to pH 5 to avoid the degradation of the Boc-group and the product is then extracted several times with portions of ethyl acetate. The solvent is removed to give the product as a white solid.

EI-MS: 238; 140 m/z; [α]^{D}=−75.68° (in MeOH, c=0.63); TLC: (Eluent: toluene/etylacetate/acetic acid 20:20:1): R_{f}=0.22; IR: s1669 cm⁻¹; s1423 cm⁻¹; m 1372 cm⁻¹; 1160 cm⁻¹; Raman: m1553 cm⁻¹; s1450 cm⁻¹; ¹H-NMR (500 MHz; DMSO): 120° C.: 1.29 ppm (s, 9H); 1.91 ppm (m, 3H); 2.34 ppm (m, 1H); 3.49 ppm (m, 2H); 5.1 ppm (m, 1H);

### EXAMPLE 3

### (S)-5-Pyrrolidin-2-yl-1H-tetrazole

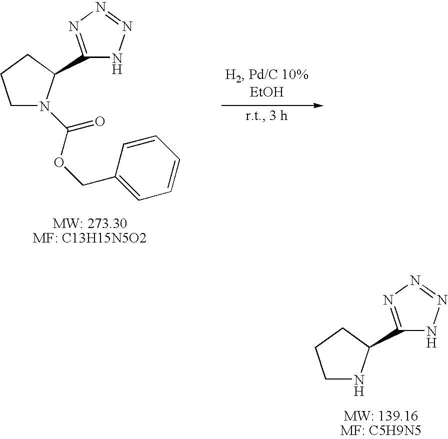

15.33 g of (S)-2-(1H-tetrazol-5-yl)-pyrrolidin-1-carboxylic acid benzyl ester (56.1 mmol) and 10% palladium on charcoal in 250 ml of ethanol are stirred under H₂ at room temperature for four to six hours. The mixture is filtered through Celite, and the Celite is washed first with ethanol and then with small amounts of acetic acid. The filtrate is evaporated to give 7.65 g of the crystalline, almost pure product. The crude product is recrystallized from hot ethanol to give the pure (S)-5-pyrrolidin-2-yl-1H-tetrazole after cooling to room temperature.

EI-MS: 139; 111; 83; 70; 43 m/z; [α]_{D}=−9.5° (in MeOH, c=0.63); TLC: (Eluent: butanol/water/acetic Acid 3:3:1): R_{f}=0.25;

¹H-NMR (500 MHz, DMSO): 2.05 (m; 3H); 2.33 (m, 1H); 3.26 (m; 2H); 4.77 (dd; 1H); 9.19 ppm (NH2: zwitterionic form;

¹³C-NMR: 23.2 ppm; 29.9 ppm; 44.3. ppm; 54.2 ppm; 171.1 ppm. Zwitterionic form confirmed by X-ray

### EXAMPLE 4

### (2S)-1-Pyrrolidinecarboxylic acid-2-(1H-tetrazol-5-yl)-phenylmethyl ester

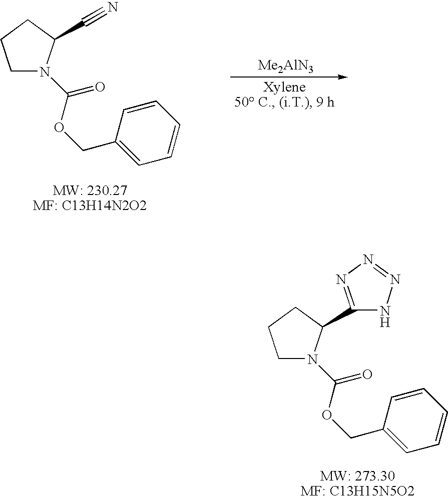

390 mg of granular sodium azide (6 mmol) are added, at 0° C., to a solution of 6 ml of dimethyl aluminum chloride (6 mmol, 1M in hexane), and the mixture is stirred at room temperature for four to seven hours. 921 mg of (2S)-pyrrolidinecarboxylic acid-2-cyano-phenylmethyl ester (4 mmol) in 2 ml of toluene are added, at 0° C., to the azide, and the mixture is warmed at 40° C. (ex.T.) 37° C. (i. T.), and stirred over the night. To destroy excess of azide, the mixture is added drop wise, at 0° C., to a solution of 15 ml of NaOH (5%; 18 mmol) containing 1.24 g of sodium nitrite (18 mmol). HCl (6N) are slowly added under cooling at 0° C., to adjust the pH to 2.5. The product is extracted with ethyl acetate. The organic phase is then extracted with bicarbonate (15%) to the aqueous phase. The aqueous phase is treated, at 0° C., with HCl (6N) to pH 2.5, and the product extracted with ethyl acetate. The solvent is removed to give pure product, which solidifies in vacuum as a white crystals.

M.p.: 84-86° C., exothermic point: 204° C.-291.38° C. with maximum at 253° C. EI-MS: 274; 272; 230; HPLC: Hewlett Packard, solvents. H₃PO₄ (0.5%), acetonitrile; flow: 2 ml/min; injection: 5.0 μl; wavelength 220 nm, 40° C. Column: Merck, Chromolith Performance RP-18e 100-4.6 mm: 5.465 min; TLC: (Eluent: toluene/etylacetate/acetic Acid 20:20:1): R_{f}=0.22; UV: (in acetonitrile, 0.10000 g/L) 236.75 nm min, 257.89 nm max; IR: 3000-2400 cm⁻¹; s1694 cm⁻¹; s1443 cm⁻¹; s1404 cm⁻¹; s1132 cm⁻¹; Raman: s1005 cm⁻¹; ¹H-NMR: (500 MHz; d₆-DMSO) 2.1 ppm (m, 1H); 2.28 ppm (m, 2H); 2.82 ppm (m, 1H); 3.56 ppm (m, 2H), 4.98 ppm (m, 2H); 5.18 ppm (m, 1H); 7.21 ppm (m, 5H);

### EXAMPLE 5

### (2R)-1-Pyrrolidinecarboxylic acid-2-(1H-tetrazol-5-yl)-phenylmethyl ester

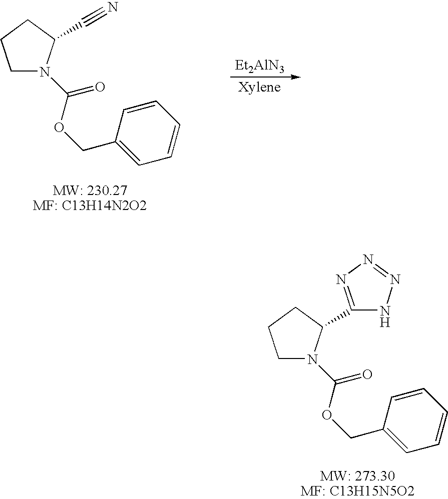

### Method: Et₂AlN₃

390 mg of granular sodium azide (6 mmol) are added to a cold solution of 2.22 ml of diethyl aluminum chloride (6 mmol, 2.7 M in xylene), and the mixture is stirred at room temperature for four to seven hours. 921 mg of (2R)-pyrrolidinecarboxylic acid-2-cyano-phenylmethyl ester (4 mmol) are added, at 0° C., to the azide, and the mixture is warmed at 54° C. (ex.T.) 50° C. (i. T.), and stirred for 6 to 10 hours.

To destroy excess of azide, the mixture is added drop wise, at 0° C., to a solution of 8.5 ml of NaOH (7%; 15 mmol) containing 1.035 g of sodium nitrite (15 mmol). HCl (6N) are slowly added under cooling at 0° C., to adjust the pH to 2.5. The product is extracted with ethyl acetate. The solvent is removed to give 1.40 g of crude product. The crude is dissolved in ethyl acetate. The product is then extracted with potassium carbonate (10%) to the aqueous phase. The aqueous phase is treated, at 0° C., with HCl (6N) to pH 2.5, and the product extracted with ethyl acetate. The solvent is removed to give an 790 mg of product as a colorless oil.

Analysis: the reaction is followed via HPLC: a sample of the mixture (c.a. 0.15 ml) is quenched with 0.5 ml of HCl 1N; 0.2 ml of acetonitrile are added, and the organic phase is checked with a HPLC

### EXAMPLE 6

### (2S)-1H-Tetrazole-5-2-pyrrolidinyl

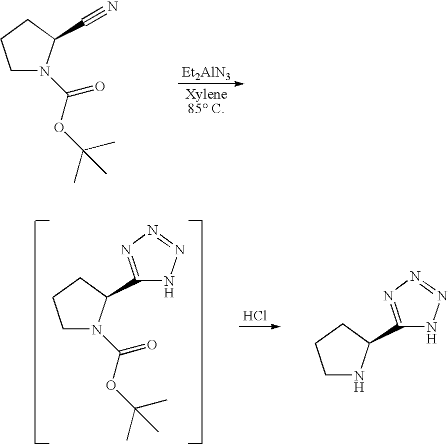

1.820 g of granular sodium azide (28 mmol) are added, at 0° C., to solution of 10.3 ml of diethyl aluminum chloride (28 mmol, 2.7 M in xylene), and the mixture is stirred at room temperature for four to seven hours. 3.920 g of 2-Cyano-pyrrolidine-1-carboxylic acid t-butyl ester (20 mmol) in 2.5 ml of toluene are added, at 0° C., to the azide, and the mixture is warmed at 50° C. (ex.T.) 45° C. (i. T.), and stirred for 8 to 14 hours.

The mixture is quenched drop wise, at 0° C., with HCl (6N) to pH 1, and stirred over the night. The aqueous phase is neutralized with solid potassium carbonate to pH 6.5, and evaporated. Ethanol is added, and the mixture is stirred for two to six hours. The mixture is filtered, and the solvent removed to give 2.8 g of product. The crude is crystallized from ethanol to give 2.26 g of product as a white crystal, with 81% of yield.

### EXAMPLE 7

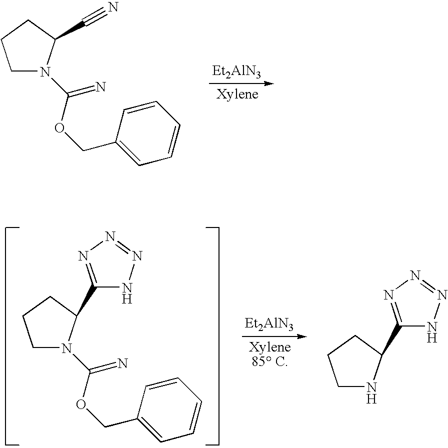

1.82 mg of granular sodium azide (28 mmol) are added, at 0° C., solution of 10.4 ml of dimethyl aluminum chloride (28 mmol, 2.7 M in xylene), and the mixture is stirred at room temperature for four to seven hours. 2.3 g of (S)-2-(1H-tetrazol-5-yl)-pyrrolidin-1-carboxylic acid benzyl ester 10 mmol) in 3 ml of toluene are added, at 0° C., to the azide. The mixture is warmed at 80° C. (i. t.), and stirred 48 hours.

The mixture is added drop wise, at 0° C., to HCl (2N). The solution is neutralized with solid potassium carbonate to pH 6.5, and the solvent is removed. Ethanol is added, and the mixture stirred for two to six hours. The mixture is filtered, and the solvent removed to give 2.66 g of white product. The product is crystallized from ethanol to give product as a white crystals.

Exothermic range: 269-365° C. (maximum: 275° C.; EI-MS: 139[M]⁺; 111[M-N₂]⁺; 83 [CH₂CN₄H]⁺; 70[CHN₄H]⁺; 43[NHCNH₂]⁺; [α]^{D}=−10.3° (in MeOH, c=0.63); TLC: (Eluent: Buthanol/water/Acetic Acid 3:3:1): R_{f}=0.25; ¹H-NMR (500 MHz, DMSO): 2.05 (m; 3H); 2.33 (m, 1H); 3.26 (m; 2H); 4.77 (dd; 1H); 9.19 ppm (NH2: switterionic form; Zwitterionic form confirmed by X-ray

### EXAMPLE 8

### methyl-5-(S)-pyrrolidine (2-yl-tetrazole)

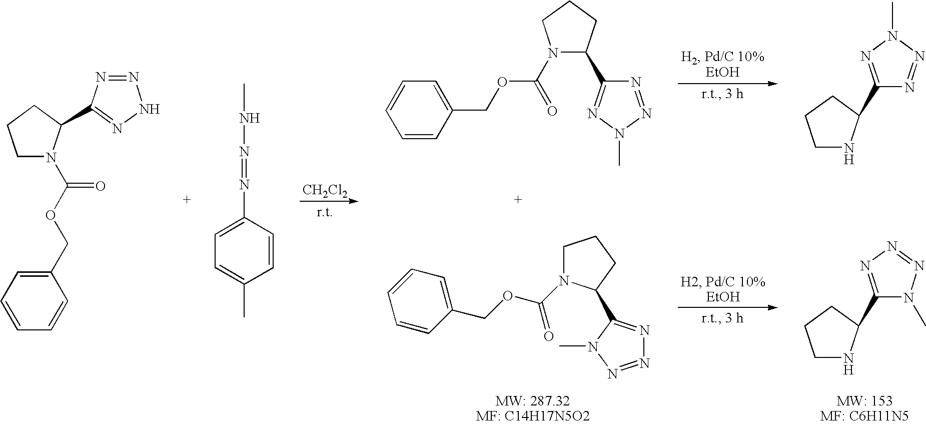

### General Procedure

### Methylation step: preparation of (S)-2-(methyl-tetrazol-5-yl)-pyrrolidine-1-carboxylic acid benzyl ester: N1, and N2-isomers

A 50 ml, three necked round-bottomed flask, is charged, at 0° C., with 2.73 g of Z—S-2-(2H-Tetrazol-5-yl)-pyrrolidine-1-carboxylic acid benzyl ester (10 mmol) dissolved in 20 ml of methylen chloride, and 2.238 g of solid 3-methyl-1-p-tolyltriazene (15 mmol). The mixture is gradually warmed at room temperature, and stirred one and half hour. The mixture is cooled down to 0° C., and quenched with 15 ml of HCl (2N). The product is extracted twice with 15 ml portion of methylene chloride. The solvent is removed to give 3.08 g of a pink oil which contains both the N-1- and N-2-isomers. The crude is dissolved in ethyl acetate and chromatographed (eluent: hexane/ethyl acetate) to give the pure (S)-2-(methyl-tetrazol-5-yl)-pyrrolidine-1-carboxylic acid benzyl ester, N-2-isomer, and N-1-isomer as oils, with a ratio of 70:30

### Hydrogenation: preparation of methyl-5-(S)-pyrrolidine (2-yl-tetrazole)

1.5 g of (S)-2-(2-Methyl-tetrazol-5-yl)-pyrrolidin-1-carboxylic acid benzyl ester (5.23 mmol) and 0.3 g of palladium on charcoal (10%) in 30 ml of ethanol are stirred under hydrogen at room temperature for two to three hours. The catalyst is removed by filtration through celite, and the celite is washed twice with a 8 ml portions of ethanol, and then with 3 ml of acetic acid, and once with 2 ml of water. The filtrate is concentrated by rotary evaporation (45° C.; 170 to 30 mbar), and dried in vacuum at room temperature for two to five hours (3.7·10⁻¹ mbar) to give the desired 2-Methyl-5-(S)-pyrrolidine-2H-tetrazole (N-2-Isomer), as a yellow oil.

### (S)-2-(2-methyl-2H-tetrazol-5-yl)-pyrrolidine-1-carboxylic acid benzyl ester (N2-Isomer)

EI-MS: 310=[M+Na]⁺; 288=[MH]⁺; 244=[MH−CO₂]⁺; HPLC (Hewlett Packard, solvents. H₃PO₄ (0.5%), acetonitrile; flow: 2 ml/min; injection: 5.0 μl; wavelength 220 nm, 40° C. Column: Merck, Chromolith Performance RP-18e 100-4.6 mm: 8.030 min; IR: FTIR-Microscope in transmission: w3064-3033 cm⁻¹; w2957-2880 cm⁻¹; s1704 cm⁻¹; s1412 cm⁻¹ m1116 cm⁻¹; m740 cm⁻¹; w699 cm⁻¹; UV: 0.1 g/L in ethanol: a) λₘₐₓ258 mn; b) λₘₐₓ205 nm ¹H-NMR: (DMSO, 400 MHz) 1.95 ppm (m; 3H); 2.35 ppm (m; 1H); 3.5 ppm (m; 2H); 4.25 ppm (s, CH₃) and 4.28 ppm (s; CH₃) rotamers; 4.97 ppm (dd; 1H); 5.15 ppm (m, 2H); 7.00 ppm (br, 1H); 7.27 ppm (m; 4H);

### (S)-2-(1-methyl-1H-tetrazol-5-yl)-pyrrolidine-1-carboxylic acid benzyl ester (N1-Isomer)

EI-MS: 310=[M+Na]⁺; 288=[MH]; HPLC (Hewlett Packard, solvents. H₃PO₄ (0.5%), acetonitrile; flow: 2 ml/min; injection: 5.0 μl; wavelength 220 nm, 40° C. Column: Merck, Chromolith Performance RP-18e 100-4.6 mm 7.080 min; IR: FTIR-Microscope in transmission: w3056 cm⁻¹; w2900 cm⁻¹; s1703 cm⁻¹; m1110 cm⁻¹; m751 cm⁻¹; w704 cm⁻¹; Raman: s1002 cm⁻¹; ¹H-NMR: (DMSO, 400 MHz) 2.02 ppm (m; 2H); 2.17 ppm (m; 1H); 2.32 ppm (m; 1H); 2.32 ppm (m; 1H); 3.54 ppm (m; 2H); 3.81 ppm (s, CH₃) and 4.10 ppm (s; CH₃) rotamers; 4.97 ppm (dd; 2H); 5.21 ppm (m, 2H); 6.99 ppm (br, 1H); 7.33 ppm (m; 4H)

### 2-Methyl-5-(S)-pyrrolidine-2H-tetrazole (N2-Isomer): (oil)

HPLC (Hewlett Packard, solvents. H₃PO₄ (0.5%), acetonitrile; flow: 2 ml/min; injection: 5.0 μl; wavelength 220 nm, 40° C. Column: Merck, Chromolith Performance RP-18e 100-4.6 mm 0.803 min; ¹H-NMR: (DMSO, 400 MHz) 1.85 ppm (m, 1H); 1.96 ppm (m, 1H), 2.18 ppm (m, 1H), 2.97 ppm (m, 2H), 4.35 ppm (m, 3H), 4.50 ppm (dd, 1H; J³=7.4 Hz);

### 1-Methyl-5-(S)-pyrrolidine-1H-tetrazole (N1-Isomer)

Mp: 148-150° C.; HPLC (Hewlett Packard, solvents. H₃PO₄ (0.5%), acetonitrile; flow: 2 ml/min; injection: 5.0 μl; wavelength 220 nm, 40° C. Column: Merck, Chromolith Performance RP-18e 100-4.6 mm 0.874 min; ¹H-NMR: (DMSO, 400 MHz) 1.75 ppm (m, 1H); 1.85 ppm (m, 1H), 2.13 ppm (m, 2H), 2.30 ppm (br, NH); 2.76 ppm (m, 1H); 2.92 ppm (m, 1H), 4.08 ppm m, 3H), 4.53 ppm (dd, 1H; J³=7.7 Hz);

### EXAMPLE 9

### 2-tButyl-5-(R)-Pyrrolidin-2-yl-2H-tetrazole

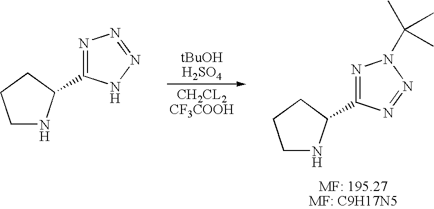

A 100 ml, three necked-round bottomed flask, is charged at 0° C., with 5.56 g of R-5-pyrrolidine-2-yl-1H-tetrazole (40 mmol) and 34.2 ml of trifluoro acetic acid (444 mmol). Then 2.6 ml of sulfuric acid (95-97%), and 5.92 g of t-butanol (80 mmol) in 8 ml of methylene chloride are added. The mixture is stirred at room temperature over the night. The mixture is quenched with 20 ml of ice-water, and the product is extracted three times with 20 ml portion of methylene chloride. The aqueous phase is treated with solid potassium carbonate until pH 8.5, and re-extracted twice with 20 ml portion of methylen chloride. The combined organic phase is washed three times with 25 ml portion of NaOH (0.5N). The solvent is removed to give the 2-tButyl-5-(R)-Pyrrolidin-2-yl-2H-tetrazole as a yellow oil.

### 2-tButyl-5-(R)-Pyrrolidin-2-yl-2H-tetrazole

pKa=8; EI-MS: 218=[M+Na]⁺; 196=[MH]⁺; 70=[C₄H₈N]⁺; IR: FTIR-Microscope in transmission: m3328 cm⁻¹; s2983 cm⁻¹; w1500 cm⁻¹; m1025 cm⁻¹; ¹H-NMR: (CDCl₃, 400 MHz) 1.69 ppm (s, 9H); 1.9 ppm (m, 3H), 2.22 ppm (m, 1H), 2.39 ppm (br, NH), 2.97 ppm (m, 1H), 3.15 ppm (m, 1H), 4.45 ppm (dd, 1H; J³=7 Hz);

### EXAMPLE 10

### (R)-2-(2-tButyl-2H-tetrazol-5-yl)pyrrolidinium chloride

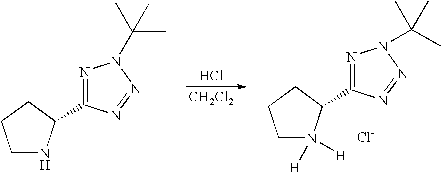

A 5 ml round bottomed flask, is charged at 0° C., with 195 mg of 2-tBu-5-(R)-2-pyrrolidine-2-yl-2H-tetrazole (1 mmol) in 2 ml of methylene chloride, and 0.5 ml of HCl (2N) (1 mmol). The mixture is stirred five minutes at room temperature, and then the solvent is removed to obtain the (R)-2-(2-tButyl-2H-tetrazol-5-yl)pyrrolidinium chloride as a pink crystalline material.

### (R)-2-(2-tButyl-2H-tetrazol-5-yl)pyrrolidinium chloride

Mp=160-164° C.; degradation starting at 136° C.; EI-MS: 196=[M+H]⁺; 140=[MH-tBu]⁺; 70=[C₄H₈N]⁺; IR: FTIR-Microscope in transmission: s2980-2458 cm⁻¹; s1410 cm⁻¹; s1375 cm⁻¹; ¹H-NMR: (DMSO, 400 MHz) 1.75 ppm (s, 9H), 2.23 ppm (m, 2H), 2.43 ppm (m, 1H), 2.55 ppm (m, 1H), 3.63 ppm (m, 2H), 5.19 ppm (m, 1H), 9.81 ppm (br, 1H), 10.9 ppm (br, 1H)

### EXAMPLE 11

### R-5-pyrrolidine-2-yl-1H-tetrazole sodium salt

### Method A:

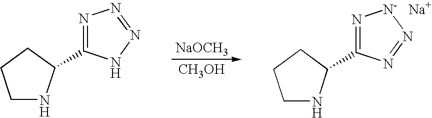

A 25 ml round bottomed flask, is charged, at r.t., with 417 mg of R-5-pyrrolidine-2-yl-1H-tetrazole (3 mmol) in 6 ml of methanol. 170 mg of sodium methylate (3 mmol) are added, the resulting solution is stirred one hour. The solvent is removed, and the product is dried in vacuum at r.t. for three hours

### Method B:

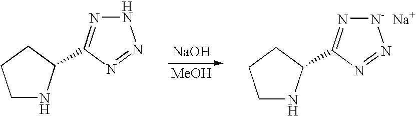

A 10 ml flask is charged, at r.t., with 417 mf of (R)-5-pyrrolidin-2-yl-2H-tetrazole (3 mmol) dissolved in 3 ml of NaOH (1N) and 1 ml of methanol. The colorless solution is stirred for 20 minutes at room temperature, and then the solvent is removed with the rotary evaporator to give 484 mg of white crystalline material. The product is re dissolved in 1.5 ml of methanol at 60° C., and the colorless solution is treated with 2.5 ml of diethylether. The solution is first cooled at 0° C., and the product crystallized after standing three hours at r.t., in an open flask. The product is filtered and washed with 1 ml of diethylether give a white crystalline material.

### R-5-pyrrolidine-2-yl-1H-tetrazole sodium salt

Mp: 62-64° C.; IR: FTIR-Microscope in transmission: s3375-2500 cm⁻¹; s1606 cm⁻¹; s1440 cm⁻¹; ¹H-NMR (DMSO; 600 MHz): 1.9 ppm (m, 3H); 2.17 ppm (m, 1H); 3.10 ppm (m, 2H); 4.55 ppm (dd, 1H, J³=7.3 Hz, 7.5 Hz); ¹³C-NMR (DMSO; 150 MHz): 24.03 ppm; 31.02 ppm; 45.05 ppm; 54.70 ppm; 166.30 ppm.

### EXAMPLE 12

### 2-(1-Methyl-1-phenyl-ethyl)5-(R)-pyrrolidin-2-yl-2H-tetrazole

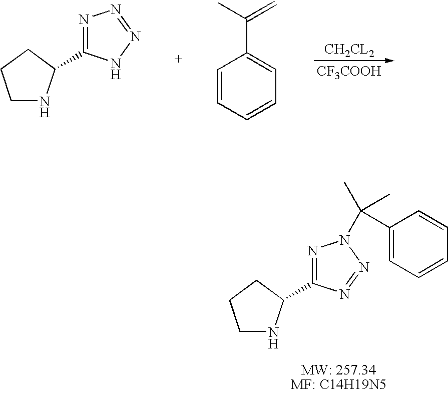

A 50 ml, three necked round-bottomed flask is charged, at r.t. under an argon atmosphere, with 4.16 g of R-5-pyrrolidine-2-yl-1H-tetrazole (30 mmol) and 4 ml of trifluoro acetic acid to obtain an yellow solution. The solution is diluted with 20 ml of methylene chloride, and then 4.55 ml of α-methylstirene (35 mmol) are added at r.t., in two portion over a period of five minutes. The homogeneous yellow mixture is stirred at r.t. for 24 hours. The mixture is transferred into a separatory funnel, and washed four times with 20 ml portion of NaOH N), and once with 20 ml of water to remove the trifluoro acetic acid. The organic phase is evaporated to give an oil which crystallize by standing twenty minutes at room temperature. The white crystalline material is washed with a small amount of hexane to remove the excess of methylstyrole, to give the pure product.

### 2-(1-Methyl-1-phenyl-ethyl)5-(R)-pyrrolidin-2-yl-2H-tetrazole

Mp: 38-40° C.; HPLC: (Hewlett Packard, solvents. H₃PO₄ (0.5%), acetonitrile; flow: 2 ml/min; injection: 5.0 μl; wavelength 220 nm, 40° C. Column: Merck, Chromolith Performance RP-18e 100-4.6 mm: 4.8 min; EI-MS: 258 [MH]⁺; 140[MH-Cumyl]⁺; 119 [Cumyl]⁺; IR: FTIR-Microscope in transmission: m3267 cm⁻¹; s2950 cm⁻¹; w1600 cm⁻¹; s1497 cm⁻¹; s1448 cm⁻¹; s766 cm⁻¹; s697 cm⁻¹; ¹H-NMR: (DMSO, 400 MHz) 1.98 ppm (m; 3H, J=7.5 Hz); 2.17 ppm (s; 6H); 2.2 ppm (s; 1H); 2.27 ppm (m; 1H); 3.04 ppm (m; 1H); 3.19 ppm (m, 1H) 4.51 ppm (dd; 1H, J=7.5 Hz); 7.10 ppm (m, 2H); 7.31 ppm (m, 3H); ¹³C-NMR: (DMSO, 150 MHz) 25.3 ppm; 28.6 ppm; 31.3 ppm; 46.2 ppm; 53.16 ppm; 67.8 ppm; 124.5 ppm; 127.6 ppm; 128.6 ppm; 144.2 ppm; 168.8 ppm. Structure confirmed by X-ray analysis.

### EXAMPLE 13

### Isopropyl-5-(R)-pyrrolidin-2-yl-1H-tetrazol

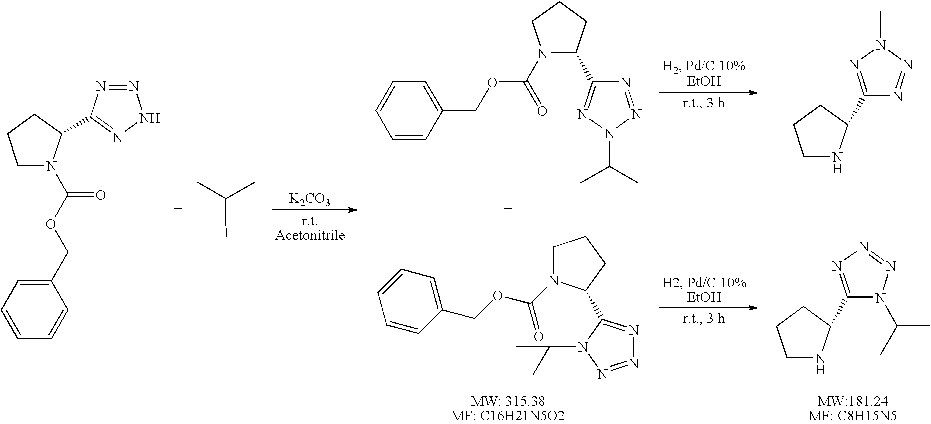

### General Procedure

### Alkylation's step: preparation of (R)-isopropyl-tetrazole-5-yl)-pyrrolidine-1-carboxylic acid benzyl ester

A 200 ml, three necked bottomed flask, equipped with a mechanic stirring apparatus, is charged, at r.t., with 12.3 g of Z—R-2-(2H-Tetrazol-5-yl)-pyrrolidine-1-carboxylic acid benzyl ester (45 mmol), in 130 ml of acetonitrile, and 24.84 g of potassium carbonate (180 mmol). After five minutes, 9.28 ml of 2-iodopropane are added (90 mmol), and the mixture is stirred for three days. The mixture is transferred into a 500 ml flask, and the acetonitrile is removed with the rotary evaporator. 40 ml of water are added (pH 8.5), and the product is extracted four times with 50 ml portion of ethyl acetate. The combined organic phase is washed once with 40 ml of water. The solvent is removed to give an orange oil which contains both the N1- and the N2-isomers. The crude is chromatographed (eluent: ethyl acetate/hexane 1:3) to give the pure (R)-isopropyl-tetrazole-5-yl)-pyrrolidine-1-carboxylic acid benzyl ester, N-1 isomer, and the pure N2-isomer fractions in a ratio of ca 30:70

### Hydrogenation: preparation of isopropyl-5-(R)-pyrrolidine (2-yl-tetrazole)

0.73 g of (R)-2-(2-isopropyl-2H-tetrazol-5-yl)-pyrrolidin-1-carboxylic acid benzyl ester (2.32 mmol) and 80 mg of palladium on charcoal (10%) in 15 ml of ethanol are stirred under hydrogen at room temperature for 8 hours. The catalyst is removed by filtration through celite, and the celite is washed twice with a 10 ml portions of ethanol, and then with 10 ml of acetic methylen chloride. The filtrate is concentrated by rotary evaporation (45° C.; 170 to 30 mbar), and dried in vacuum at room temperature for two to five hours (3.7·10⁻¹ mbar) to give the desired pure 2-isopropyl-5-(R)-pyrrolidin-2-yl-2H-tetrazol, as a yellow oil.

### (R)-2-(2-isopropyl-2H-tetrazol-5-yl)-pyrrolidin-1-carboxylic acid benzyl ester; N2-isomer

EI-MS: 338=[M+Na]⁺; 316=[MH]⁺; 272=[MH−CO₂]⁺; IR: FTIR-Microscope in transmission: m3033-2880 cm⁻¹; s1707 cm⁻¹; s1412 cm⁻¹; s1356 cm⁻¹; m1180 cm⁻¹; m1116 cm⁻¹; m1025 cm⁻¹; Raman Liquid: m3062-2880 cm⁻¹; m1029 cm⁻¹; s1003 cm⁻¹; UV: 0.1 g/L in ethanol: a) λₘₐₓ 258 nm; Abs=0.0141; ε (1%, 1 cm)=14; b) λₘₐₓ 205 nm; Abs=0.4191; ε (1%, 1 cm)=419; ¹H-NMR: (DMSO, 400 MHz) 1.51 ppm (m; 6H, two rotamers, J³=6.5 Hz); 1.95 ppm (m; 3H); 2.33 ppm (m; 1H); 3.53 ppm (m, 2H), 4.95 ppm (m; 3H); 5.20 ppm (dd; 1H, J³=8 Hz); 7.02 ppm (m, 1H); 7.25 ppm (m, 2H); 7.36 ppm (m; 2H);

### (R)-2-(1-isopropyl-1H-tetrazol-5-yl)-pyrrolidin-1-carboxylic acid benzyl ester; N1-Isomer

EI-MS: 338=[M+Na]⁺; 316=[MH]⁺; IR: FTIR-Microscope in transmission: w3064 cm⁻¹; m2983-2882 cm⁻¹; s1703 cm⁻¹; w1587-1499 cm⁻¹; s1414 cm⁻¹; s1357 cm⁻¹; m1123 cm⁻¹; w1028 cm⁻¹; Raman Liquid: m3063-2945 cm⁻¹; s1003 cm⁻¹; UV: 0.1 g/L in ethanol: a) λₘₐₓ258 nm Abs=0.007; ε (1%, 1 cm)=7; b) λₘₐₓ205 nm Abs=0.3200; ε (1%, 1 cm)=320; ¹H-NMR: (DMSO, 400 MHz): 1.30 ppm (d, 6H; J=6.5 Hz rotamers); 1.53 ppm (d, 6H, J=6.5 Hz rotamers), 1.90 ppm (m, 2H), 2.2. ppm (m, 2H), 3.58 ppm (m, 2H); 4.90 ppm (1H), 5.31 ppm (m, 1H), 6.97n ppm (m, 1H), 7.35 ppm (m, 4H)

### 1-Isopropyl-5-(R)-pyrrolidin-2-yl-1H-tetrazol: N1 isomer

¹H-NMR: (DMSO, 400 MHz): 1.51 ppm (d, 6H rotamers J³=6.6 Hz); 1.56 ppm (d, 6H rotamers, J³=6.5 Hz); 2.0 ppm (m, 2H); 2.29 ppm (m, 2H); 5.03 ppm (m, 1H, J³=6.5 Hz); 5.16 ppm (dd, 1H, J³=7.5 Hz)

### 2-Isopropyl-5-(R)-pyrrolidin-2-yl-2H-tetrazol: N2 isomer

EI-MS: 182=[MH]⁺; 113=[MH−C4H7N]⁺; 70=[C₄H₈N]⁺; IR: FTIR-Microscope in transmission: m3342 cm⁻¹; s2983-2940 cm⁻¹; s1458 cm⁻¹; s1063 cm⁻¹; Raman: s1450 cm⁻¹; ¹H-NMR: (DMSO, 400 MHz): 1.55 ppm (d, 6H, J³=6.8 Hz); 1.85 ppm (, 3H), 2.15 ppm (m, 2H), 3.1 ppm (br, 1H); 4.33 ppm (br NH), 4.43 ppm (dd, 1H, J³=7.3 Hz; 6.8 Hz), 5.07 ppm (sep, 1H, J³=6.7 Hz); ¹³C-NMR: (DMSO, 400 MHz) 21.84 ppm; 23.17 ppm; 30.96 ppm; 46.04 ppm; 53.02 ppm; 55.73 ppm; 167.32 ppm.

### EXAMPLE 14

### 2-[(1-Methyl-1-phenyl-ethyl)-2H-tetrazol-5-yl]pyrrolidinium saccharinate

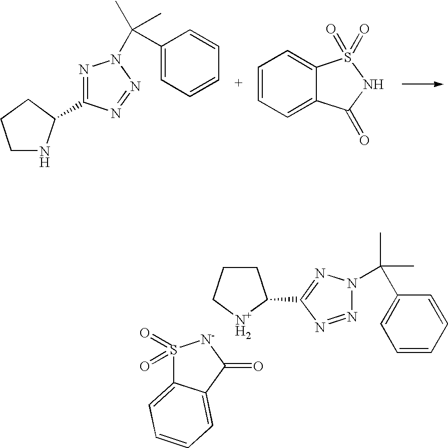

A 10 ml round bottomed flask is charged with 128 mg of 2-(Methyl-1 phenyl-ethyl)-5-(R)-pyrrolidin-2-yl-2H-tetrazole (0.5 mmol), and 91 mg of o-benzoic acid sulfimide (0.5 mmol) in 2 ml of methylene chloride to obtain a colorless solution. The solvent is removed to obtain a colorless oil which is dissolved in 0.5 ml of methylene chloride and 1 ml of diethyl ether. The product crystallized after standing at r.t. for three hours into an open flask. The white crystalline material is filtered, and washed with 1 ml of diethyl ether/methylen chloride (3:1) to give 110 mg of product. The mother liquor is lead standing over the night into a closed flask. The product is filtered to give 110 mg as second crop.

### 2-[(1-Methyl-1-phenyl-ethyl)-2H-tetrazol-5-yl]pyrrolidinium saccharinate

Mp: 108-111° C.; EI-MS: 258 [M_{B}]⁺; 140 [M_{B}-Cumyl]⁺; 182 [M_{A}]⁻; IR: Microscope in transmission: w 3060 cm⁻¹; w2990-2900 cm⁻¹; w 2800-2300 cm⁻¹; s1651 cm⁻¹; s1152 cm⁻¹ (SO2); s770 cm⁻¹; s759 cm⁻¹; UV: in ethanol, c=0.1 g/L: a) λₘₐₓ 201.19 nm; b) λₘₐₓ=264.07 nm; ¹H-HNMR (DMSO, 400 Mz): 2.13 ppm (s, 6H); 2.07 ppm (m, 2H); 2.31 ppm (m, 2H); 3.35 ppm (m, 2H); 5.04 ppm (dd, 1H, J³=8 Hz), 7.13 ppm (m, 2H), 7.30 ppm (m, 1H); 7.34 ppm (m, 2H); 7.59 ppm (m, 3H), 7.66 ppm (m, 1H); 9.48 (br, 2H, NH₂⁺), ¹³C-NMR (DMSO, 150 MHz): 23.06 ppm; 28.66 ppm; 28.92 ppm; 45.32 ppm; 53.19 ppm; 69.00 ppm; 119.17 ppm, 122.56 ppm; 124.7 ppm; 127.94 ppm; 128.6 ppm, 131.16 ppm, 131.67 ppm; 134.48 ppm, 143.51 ppm; 145.00 ppm; 161.81 ppm; 167.53 ppm

### EXAMPLE 15

### R-5-pyrrolidine-2-yl-1H-tetrazole potassium salt

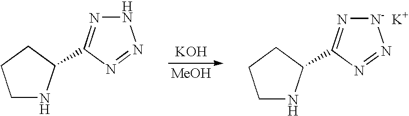

A 10 ml flask is charged, at r.t., with 347 mf of (R)-5-pyrrolidin-2-yl-2H-1-tetrazole (2.5 mmol), and 140 mg of KOH (2.5 mmol) in 4 ml of methanol. The colorless solution is stirred for 20 minutes at room temperature, and then the solvent is removed with the rotary evaporator to give 440 mg of white crystalline material. The product is re dissolved in 1.5 ml of methanol at 60° C., and the colorless solution is treated with 2.5 ml of diethylether. The solution is first cooled at 0° C., and the product crystallized after standing three hours at r.t., in an open flask. The product is filtered and washed with 1 ml of diethylether give a white crystalline material.

### R-5-pyrrolidine-2-yl-1H-tetrazole potassium salt

Mp=58-62° C.; ¹HNMR (400 MHz, DMSO) 1.69 ppm (m, 3H), 192 ppm (m, 1H), 2.66 ppm (m, 1H), 3.02 ppm (m, 1H), 4.09 ppm (dd, 1H, J³=6.8 Hz)

### EXAMPLE 16

### R-5-pyrrolidine-2-yl-1H-tetrazole palladium(II) complex

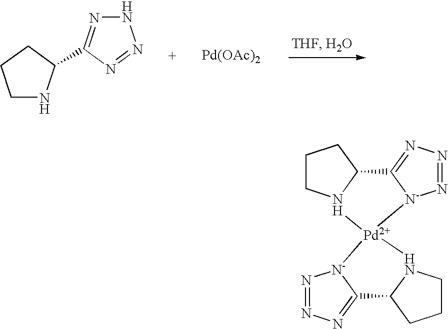

A 10 ml, one necked round bottomed flask is charged, at r.t., with 139 mg of (R)-5-pyrrolidin-2-yl-2H-tetrazole (1 mmol), and 112 mg of palladium acetate (0.5 mmol) dissolved in 1.5 ml of a solution of THF/water (2:1). The mixture is warmed under stirring at 50° C. The with product crystallize after standing at room temperature for two hours. The product is filtered and dried in vacuum to removed the acetic acid formed.

### R-5-pyrrolidine-2-yl-1H-tetrazole palladium(II) complex

Mp: 268-271° C. (decomposition); IR: Microscope in Transmission: s3132 cm⁻¹; m1404 cm⁻¹, s1129 cm⁻¹; UV: in ethanol, c=0.1 g/L: a) λₘₐₓ=203.74 nm; b) λₘₐₓ=287.96 nm; ¹H-NMR (400 MHz, DMSO): 1.87 ppm (m, 3H); 2.44 ppm (m, 1H); 3.44 ppm (m, 2H); 4.51 ppm (dd, 1H, J³=7.3H); 7.57 ppm (m, NH⁺); ¹³C-NMR (DMSO, 150 MHz): 26.38 ppm, 31.00 ppm; 52.22 ppm, 56.86 ppm; 167.24 ppm

### EXAMPLE 17

### (R)-2-(2-tButyl-2H-tetrazol-5-yl)pyrrolidinium trifluoro acetate

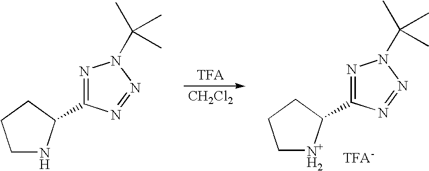

A 10 ml round bottomed flask, is charged at r.t., with 390 mg of 2-tBu-5-(R)-2-pyrrolidine-2-yl-2H-tetrazole (2 mmol) in 2 ml of methylene chloride, then 228 mg trifluoroacetic acid (2 mmol) are added. The mixture is stirred five minutes at room temperature, and then the solvent is removed to obtain the product as a brown crystalline material.

### (R)-2-(2-tButyl-2H-tetrazol-5-yl)pyrrolidinium trifluoro acetate

Mp=87-90° C.; EI-MS: 196=[MH-TFA]⁺; 140=[MH-tBu-TFA]⁺; IR: FTIR-Microscope in transmission: m2990-2600 cm⁻¹; s1674 cm⁻¹; w1430 cm⁻¹; s1200-1140 cm⁻¹; m798 cm⁻¹; m722 cm⁻¹; ¹H-NMR: (DMSO, 400 MHz) 1.72 ppm (s, 9H), 2.26 ppm (m, 2H), 2.45 ppm (m, 1H), 2.55 ppm (m, 1H), 3.63 ppm (m, 2H), 5.17 ppm (dd, 1H, J³=7 Hz, 7.3 Hz), 8.99 ppm (br, 1H), 10.33 ppm (br, 1H). Structure confirmed by X-ray analysis.

### EXAMPLE 18

### R-5-pyrrolidine-2-yl-1H-tetrazole cesium salt

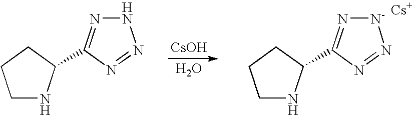

A 10 ml flask is charged, at r.t., with 417 mf of (R)-5-pyrrolidin-2-yl-2H-tetrazole (3 mmol) dissolved in 4 ml of water, and with 504 mg of cesium hydroxide monohydrate (3 mmol). The colorless solution is stirred for 20 minutes at room temperature, and then the solvent is removed with the rotary evaporator to give 810 mg of white crystalline material. The product is re dissolved in 1.5 ml of methanol at 50° C., and the colorless solution is treated with 2.5 ml of diethylether. The solution is first cooled at 0° C., and the product crystallized after standing three hours at r.t., in an open flask. The product is filtered and washed with 1 ml of diethylether give a white crystalline material.

### R-5-pyrrolidine-2-yl-1H-tetrazole cesium salt

Mp=55-58° C.; ¹H-NMR: (DMSO, 400 MHz) 1.71 ppm (m, 3H), 1.94 ppm (m, 1H), 2.70 ppm (m, 1H), 3.04 ppm (m, 1H), 4.12 ppm (dd, 1H, J³=6.8 Hz)

### EXAMPLE 19

### (R)-2-[(1-Methyl-1-phenyl-ethyl)-2H-tetrazol-5-yl]pyrrolidinium trifluoro acetate

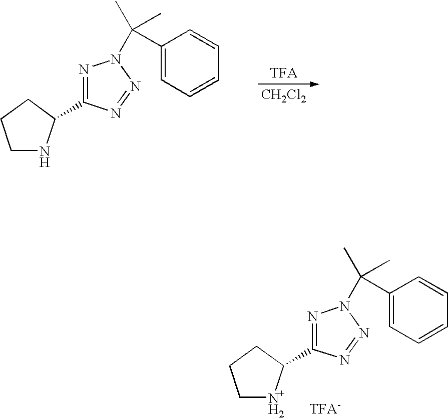

A 10 ml round bottomed flask, is charged at r.t., with 514 mg of 2-(Methyl-1-phenyl-ethyl)-5-(R)-pyrrolidin-2-yl-2H-tetrazole (2 mmol) in 2 ml of methylene chloride, then 228 mg trifluoroacetic acid (2 mmol) are added. The mixture is stirred five minutes at room temperature, and then the solvent is removed to obtain the product as a white crystalline material.

### (R)-2-[(1-Methyl-1-phenyl-ethyl)-2H-tetrazol-5-yl]pyrrolidinium trifluoro acetate

Mp: 128-130° C.; ¹H-NMR (DMSO, 400 MHz): 2.14 ppm (s, 6H); 2.20 ppm (m, 2H); 2.43 ppm (m, 1H); 2.54 ppm (m, 1H); 5.12 ppm (dd, 1H, J³=7.3 Hz; 7 Hz); 7.09 ppm (m, 2H), 7.30 ppm (m, 3H)

What is claimed is:

1. A process for the preparation of (S)-pyrrolidine-1H-tetrazole derivatives of formula 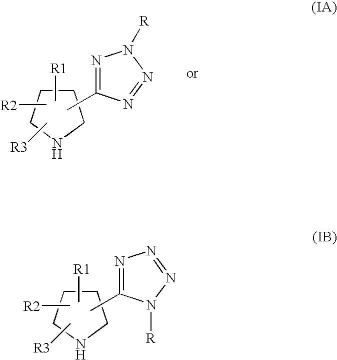
wherein
R₁, R₂ and R₃, independently of one another, represent hydrogen, halogen or an organic radical, and R is hydrogen or is selected from the group consisting of a branched C₃-C₇-alkyl, methyl that can be substituted by one, two or three substituents selected from C₁-C₇-alkyl and C₁-C₇-alkoxy; allyl that can be substituted by one, two or three substituents selected from OH, halo and C₁-C₇-alkoxy, cinnamyl that can be substituted by one, two or three substituents selected from C₁-C₇-alkyl, C₁-C₇-alkoxy and C₂-C₈-alkanoyloxy, C₁-C₃-alkyl that is mono-, di or trisubstituted by phenyl, wherein the phenyl ring is unsubstituted or substituted by one or more, e.g. two or three, substituents e.g. those selected from the group consisting of tert-C₁-C₇-alkyl or C₁-C₇-alkoxy; C₂-C₈-alkanoyloxy; aralkanoyloxy; fluorenyl; silyl such as tri-C₁-C₄-alkyl-silyl, or di-C₁-C₄-alkyl-phenyl-silyl; C₁-C₇-alkyl-sulphonyl; arylsulphonyl such as phenylsulphonyl wherein the phenyl ring is un-substituted or substituted by one or more, e.g. two or three, substituents selected from the group consisting of C₁-C₇-alkyl, C₁-C₇-alkoxy, C₂-C₈-alkanoyl-oxy; C₂-C₈-alkanoyl; benzoyl and esterified carboxy; or R is a cation;
in racemic form or as an enantiomer, a tautomer, an analog thereof or a salt thereof, comprising
(i) reacting a compound of formula 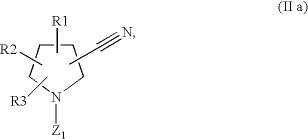
wherein variables R₁, R₂ and R₃ have the meaning as defined above,
with an azide of formula (R₄)(R₅)M-N₃ (II b), wherein R₄ and R₅, independently of another, represent an organic residue such as an aliphatic residue, an alicyclic residue, a heteroalicyclic residue; an alicyclic-aliphatic residue; a heteroalicyclic-aliphatic residue; a carbocyclic or a heterocyclic aromatic residue; an araliphatic residue or an heteroaraliphatic residue, each residue, independently of another, being unsubstituted or substituted; and M is boron or aluminium; and Z₁ represents a protecting group, and
(ii) isolating the resulting compound of formula (IA) or (IB).

2. A process for the preparation of (S)-pyrrolidine-1H-tetrazole derivatives of formula 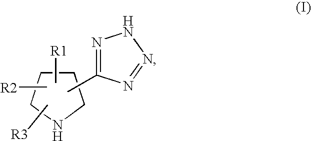
wherein
R₁, R₂ and R₃, independently of one another, represent hydrogen, halogen or an organic radical, in racemic form or as an enantiomer, a tautomer, an analog thereof or a salt thereof, comprising
(i) reacting a compound of formula 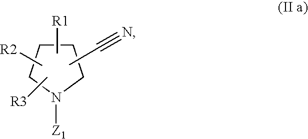
wherein variables R₁, R₂ and R₃ have the meaning as defined above, with an azide of formula (R₄)(R₅)M-N₃ (II b), wherein R₄ and R₅, independently of another, represent an organic residue such as an aliphatic residue, an alicyclic residue, a heteroalicyclic residue; an alicyclic-aliphatic residue; a heteroalicyclic-aliphatic residue; a carbocyclic or a heterocyclic aromatic residue; an araliphatic residue or an heteroaraliphatic residue, each residue, independently of another, being unsubstituted or substituted; and M is boron or aluminium; and Z₁ represents a protecting group, and
(ii) isolating the resulting compound of formula (I).

3. A process for the manufacture of a compound of formula (I), in racemic form or as an enantiomer, as a tautomer, an analog thereof or a salt thereof, wherein R represents an organic residue;
comprises
(a) reacting a compound of formula 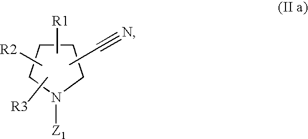
wherein variables R₁, R₂ and R₃ have the meaning as defined above and Z₁ is a protecting group,
with an azide of formula (R₄)(R₅)M-N₃ (II b), wherein R₄ and R₅ have the meaning as defined above,
(b) splitting off the protecting group in a resulting compound of formula (II c) 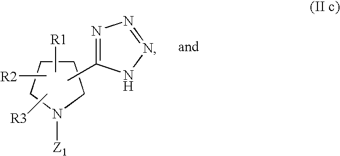
(c) isolating the resulting compound of formula (I).

4. Process according to any of claims 1 to 3 for the manufacture of a pyrrolidine-1H-tetrazole of formulae 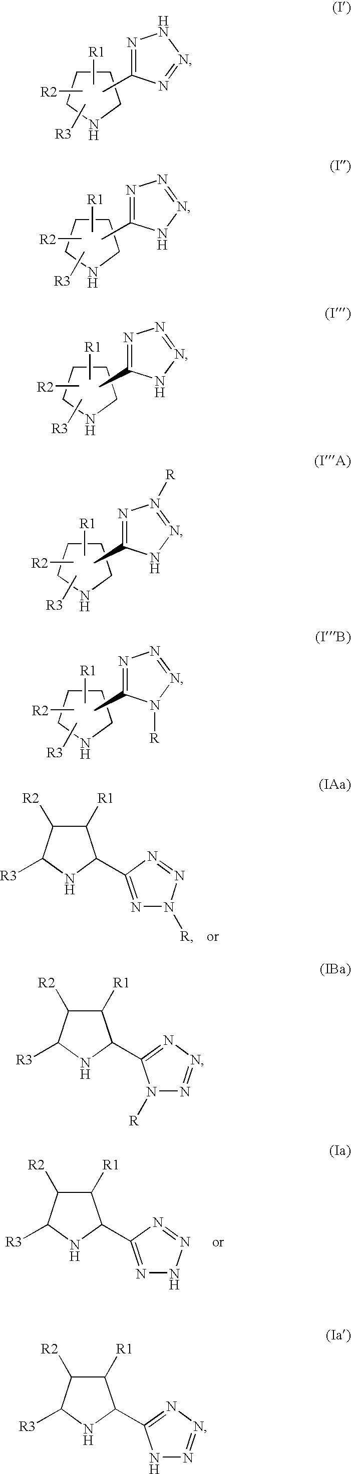 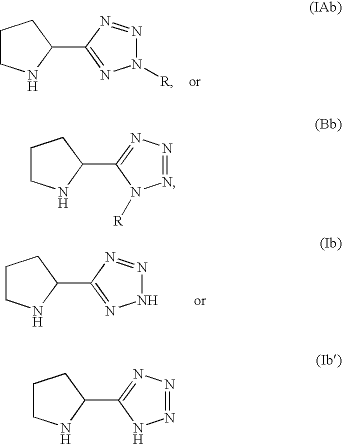
in racemic form or as an enantiomer, as a tautomer, an analog thereof or a salt thereof.

5. Process according to any one of claims 1 to 4 for the manufacture of a compound of formulae (I), (I′), (I″), (I′″), (I′″A), (I′″B), (IAa), (IBa), (I a), (I a′), (IAb), (Bb), (I b) or (I b′), wherein R₁, R₂ and R₃, independently of one another, represent hydrogen, halogen, an aliphatic residue, an alicyclic residue, a heteroalicyclic residue; an alicyclic-aliphatic residue; a heteroalicyclic-aliphatic residue; a carbocyclic or a heterocyclic aromatic residue; an araliphatic residue or an heteroaraliphatic residue, each residue, independently of one another, being unsubstituted or substituted.

6. Process according to claim 5, for the manufacture of a compound of formulae (I), (I′), (I″), (I′″), (I′″A), (I′″B), (IAa), (IBa), (I a), (I a′), (IAb), (Bb), (I b) or (I b′), wherein R₁, R₂ and R₃, independently of one another, represent hydrogen; alkyl, alkenyl or secondarily alkynyl, each of which can be interrupted by NH, substituted NH, O, or S; and each of which can be unsubstituted or substituted, for example, mono-, di- or tri-substituted; cycloalkyl and secondarily cycloalkenyl, each of which can also be substituted; alicyclic residue, wherein at least one carbon atom is replaced by NH, substituted NH, O, or S, each of which can also be substituted; alkyl, alkenyl or alkynyl that is substituted by cycloalkyl or by cycloalkenyl; a mono- or polycyclic or benzoanellated carbocyclic residue which can also be substituted; 5- or 6-membered and monocyclic radical which has up to four identical or different hetero atoms selected from nitrogen, oxygen and sulfur atoms, each of which can also be substituted.

7. Process according to claim 6, for the manufacture of a compound of formulae (I), (I′), (I″), (I′″), (I′″B), (IAa), (IBa), (I a), (I a′), (IAb), (Bb), (I b) or (I b′), wherein R₁, R₂ and R₃, independently of one another, represent hydrogen; halogen.

8. Process according to any one of claims 1 to 4 for the manufacture of a compound of formulae (I), (I′), (I″), (I′″), (I′″A), (I′″B), (IAa), (IBa), (I a), (I a′), (IAb), (Bb), (I b) or (I b′), wherein R₁ is hydrogen and R₂ and R₃, independently of one another, represent hydrogen, halogen, an aliphatic residue, an alicyclic residue, a heteroalicyclic residue; an alicyclic-aliphatic residue; a heteroalicyclic-aliphatic residue; a carbocyclic or a heterocyclic aromatic residue; an araliphatic residue or an heteroaraliphatic residue, each residue, independently of one another, being unsubstituted or substituted.

9. Process according to claim 8, for the manufacture of a compound of formulae (I), (I′), (I″), (I′″), (I′″A), (I′″B), (IAa), (IBa), (I a), (I a′), (IAb), (Bb), (I b) or (I b′), wherein R₁ is hydrogen and R₂ and R₃, independently of one another, represent hydrogen; alkyl, alkenyl or secondarily alkynyl, each of which can be interrupted by NH, substituted NH, O, or S; and each of which can be unsubstituted or substituted, for example, mono-, di- or tri-substituted; cycloalkyl and secondarily cycloalkenyl, each of which can also be substituted; alicyclic residue, wherein at least one carbon atom is replaced by NH, substituted NH, O, or S, each of which can also be substituted; alkyl, alkenyl or alkynyl that is substituted by cycloalkyl or by cycloalkenyl; a mono- or polycyclic or benzoanellated carbocyclic residue which can also be substituted; 5- or 6-membered and monocyclic radical which has up to four identical or different hetero atoms selected from nitrogen, oxygen and sulfur atoms, each of which can also be substituted.

10. Process according to claim 6, for the manufacture of a compound of formulae (I), (I′), (I″), (I′″), (I′″A), (I′″B), (IAa), (IBa), (I a), (I a′), (IAb), (Bb), (I b) or (I b′), wherein R₁ is hydrogen and R₂ and R₃, independently of one another, represent hydrogen; halogen.

11. Process according to any one of claims 1 to 4 for the manufacture of 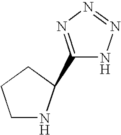
or a tautomer or salt thereof.

12. Process according to any one of claims 1 to 11, wherein an azide of formula (R₄)(R₅)M-N₃ (II b) is used, wherein M is aluminium or boron, R₄ and R₅, independently of one another, is C₁-C₈-alkyl such as methyl, ethyl, propyl, i-propyl, diisobutyl, tert-butyl or n-octyl; C₃-C₇alkenyl such as allyl or crotyl, C₃-C₇-cycloalkyl such as cyclohexyl; phenyl-C₁-C₄-alkyl such as benzyl or 2-phenethyl; phenyl-C₃-C₅alkenyl such as cinnamyl, or C₃-C₈-cycloalkyl-C₁-C₈-alkyl such as cyclopropylmethyl or cyclohexylmethyl.

13. A compound of formula 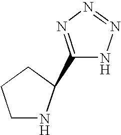
or a tautomer or salt thereof.

14. A compound of formula 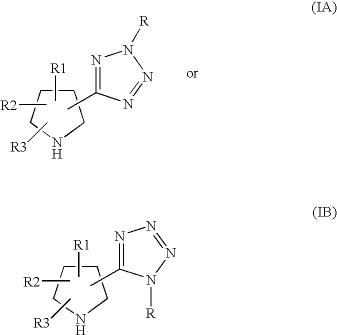
wherein
R₁, R₂ and R₃, independently of one another, represent hydrogen, halogen or an organic radical, and R is hydrogen or tert-butyl, methyl, isopropyl, benzyl, p-methoxybenzyl, 3,4-dimethoxybenzyl, 1-methyl-1-phenylethyl, triphenylmethyl, (p-methoxyphenyl)-diphenylmethyl, benzyloxymethyl, allyl and cinnamyl in racemic form or as an enantiomer, a tautomer, or a salt thereof.

15. A compound of formula 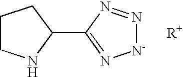
wherein
R₁, R₂ and R₃, independently of one another, represent hydrogen, halogen or an organic radical, and R is a cation, e.g. of an alkali metal or an earth alkali metal, for example Li(I), Na(I), K(I), Rb(I), Cs(I), Mg(II), Ca(II), AI(III), Pd (II), Pt(II), Cu(I), Cu(II) and Sr(II). in racemic form or as an enantiomer or a tautomer thereof.

16. Use of a compound of formulae (I), (I′), (I″), (I′″), (I a), (I a′), (I b) or (I b′) or 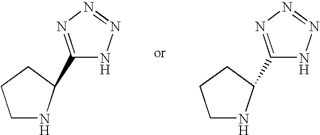
or a tautomer or salt thereof as organo catalysts in the preparation of chiral compounds for example in aldol reactions, nitroaldol reactions, mannich type reactions, α-amination reactions, α-hydroxylation reactions, michael reactions, and in the synthesis of enantiopure compounds.
